SEQUENCE LISTING (34 sequences)

SEQ ID NO: 1 - DNA, 45 bp, Artificial
  synthetic DNA
cgtgaacaggaatagtccactcgaaaatgcggattatatttacgg

SEQ ID NO: 2 - DNA, 46 bp, Artificial
  synthetic DNA
cctacaaggaacaaagaccatcggtgaccgatgtattaattttgcc

SEQ ID NO: 3 - DNA, 18 bp, Artificial
  synthetic DNA
actcattgcagcatttgc

SEQ ID NO: 4 - DNA, 22 bp, Artificial
  synthetic DNA
agacacaatgaatatcttctgg

SEQ ID NO: 5 - DNA, 25 bp, Artificial
  synthetic DNA
ttctttattaaattttttgatgcca

SEQ ID NO: 6 - DNA, 25 bp, Artificial
  synthetic DNA
aactatttttatcaatgtctcctgg

SEQ ID NO: 7 - DNA, 24 bp, Artificial
  synthetic DNA
gaactatttttatcaatgtctcct

SEQ ID NO: 8 - DNA, 47 bp, Artificial
  synthetic DNA
ggcttgcccaccattttctttatctaagattattaaaaatggcagcg

SEQ ID NO: 9 - DNA, 50 bp, Artificial
  synthetic DNA
tctgcaagaaatgttggaattgagcttttactaacaaaatcatcagggtc

SEQ ID NO: 10 - DNA, 20 bp, Artificial
  synthetic DNA
gatgatggttcagtagatgc

SEQ ID NO: 11 - DNA, 24 bp, Artificial
  synthetic DNA
ctgatatttgtttatcgacttcag

SEQ ID NO: 12 - DNA, 47 bp, Artificial
  synthetic DNA
ctgaaatgcagaggttaattgcatccaactgcttttaattcagagcc

SEQ ID NO: 13 - DNA, 47 bp, Artificial
  synthetic DNA
tcaaagaactctttcttcttgggaataaacaggttgtatcggtcatc

SEQ ID NO: 14 - DNA, 23 bp, Artificial
  synthetic DNA
tcgatatttcgttagaacatctc

SEQ ID NO: 15 - DNA, 24 bp, Artificial
  synthetic DNA
ctaagaagagttttacaaccattc

SEQ ID NO: 16 - DNA, 41 bp, Artificial
  synthetic DNA
ctccactgcgaaaagctcaacaatggacaagtctacctcaa

SEQ ID NO: 17 - DNA, 44 bp, Artificial
  synthetic DNA
gagggttctttcaaactattgcttggcttaggggtttcttcact

SEQ ID NO: 18 - DNA, 18 bp, Artificial
  synthetic DNA
attggaaaggtcgccgta

SEQ ID NO: 19 - DNA, 18 bp, Artificial
  synthetic DNA
gtaatagctgccagtgct

SEQ ID NO: 20 - DNA, 46 bp, Artificial
  synthetic DNA
acccaagataagaattctctctaatttatatcaacttgctgttcaa

SEQ ID NO: 21 - DNA, 48 bp, Artificial
  synthetic DNA
ttggacttgatagtaccaaaaacagttagcaactaaattactaccata

SEQ ID NO: 22 - DNA, 22 bp, Artificial
  synthetic DNA
tgagttatacagtatcgatctc

SEQ ID NO: 23 - DNA, 24 bp, Artificial
  synthetic DNA
tgtcatctgaaaaatttctaacgt

SEQ ID NO: 24 - DNA, 24 bp, Artificial
  synthetic DNA
cattcatcattttaagttggcgtt

SEQ ID NO: 25 - DNA, 25 bp, Artificial
  synthetic DNA
ggcctatttttatgataaacaacac

SEQ ID NO: 26 - DNA, 23 bp, Artificial
  synthetic DNA
cattcatcattttaagttggcgt

SEQ ID NO: 27 - DNA, 5882 bp, Haemophilus influenzae
cggcacagggctgataaaaaacgtctcagtctcaaacaggtgaaatttataggttatctttttataaaatatttagtaatccatcgcctattttcataaattttatgatagaattaaaacttaatatgacatacaaccggtaaaaagtggtctttttttaaattaaggctacagttttcttgtgtatgcaagataaatagatacaagatcttgttatctaaatgtcactttaccaaaaaatattttaagctaatctgttgggatatcatatgttaaaaaataaaaacatagggatcattttggctggtggcataggctctcgcatgggcctaggttacccaaaacaattttcaaaaattgcaggtaaaacagcactagaacacacaattttcatttttcaagaacataaagaaattgatgaaattatcatcgtttctgagcgtacctcttatcgtcgtattgaagatatcgtatcaaaagctggattttccaaagttaatcgtattatttttggtggtaaagaacgctctgattctactctttctgcaatcacagctcttcaagatgaaccaagaaatacgaaattaatcattcatgatgctgtacgacctttactagcgactgaaataatctctgaatgtattgcgaaattagataagtacaatgctgtagatgtggctattcctgcagttgataccattgttcatgttaataatgatacccaagaaattattaaaattcctaagcgtgctgaatactaccaaggccaaactccacaagcatttaaactaggcacgctaaaaaaagcttacgatatttatacacaaggtggcatcgaaggtacttgtgattgttctattgtgttaaaaaccctacctgaagaaagagttggtatcgtttcaggttttgaaaccaacattaaactaactcgcccagttgatctttttatcgctgataaattattccaaagccgtagtcatttttcactacgtaatatcacctctattgatcgcctatatgatatgaaagatcaggtattagttgttattggtggaagctatggtattggcgcgcatattatcgatgttgcaaaaaaatttggaattaaaacatatagccttagtcgttcaaatggtgtcgatgttggtgatgttaagtctattgaaaaagcattcgcaggaatttacggaaaagaacacaaaatagaccatatcgtaaatactgctgcggtgttaaatcataaaacgttagcatcaatgtcttatgaagaaattgtaactagtatcaatgtaaactacacgggcatgattaatgctgtgataacagcttatccttacttaaaacaaactcatggtagttttttaggtttcacatcaagctcgtatacacgaggccgtccattctatgctatttactcttctgcaaaagcagctgtggtaaacttaactcaagccatctctgaagaatggttaccggataatattaaaattaactgcgttaatccagagagaacaaaaacaccaatgagaaccaaagcattcggtattgaaccagaaggcacactacttgatcccaaaacggtagcatttgcttctcttactgtactcgctagcagagagactggtaacatcatcgatgttgtattaaaagatgaagaatatatttcccatattttagctgatctttataaataaatttgagtgagtctgttatggctaatacaccctattttatttatcttgacaccaaaattattggcgcagtaaagcaaacaattacctttttcgaacatggtgtgttttctcgtggaaacacaaccattcttgtgaaaaagtataagcataagtcagcaaaaataattgagcgagctttaattaaagcctcgctcaattatcattttgtcaatgccgcctaccttgatcgattaacagaaggtgttattttttacccatttaatgcacagtcaaattgccgagcagtagcaaaccgtaaacttactcatatttttatcactcatggtgaaagcaataaaatcacctcagttaagcctattactcgcatttatgatcatgttattactgcaggtaaagctggtgtagatcgctttctttcccataaaattttctctcaatatgatgttgatacaggcagaatcattcctatgggagatacatttattgggaaaacaggactagattgtacaggtaaaggcactccggtcatcttttatgctccaacatgggaaggcggtattgagcaagaaaattactctagccttgctcatataaatcaagttgtggcaaccatcttacaacttagcgaatactatcaagtgaagcatgtcgccattcgtccacacctaaacacaggacaccgcagaccagaatatcacaattttttattacgaataatagaaagcttacaagcaaaatcactaaaactcgtacttttcaaaccttacttgaatttttcttttgcccaagaatggaaattaaagcggagagatgttgtttttaaaacaaacttcagtgagtttagtgctgtagttggcctgtgcgatatttctgcaatggaaactcaattacttaacgaaaatatttattactacttattctgctctgaagagtacaagcagtatctattgacgttaaagaacagtgaatactatcaaaataacgcattaatctttgattgcggggaattgattccttctcaagaattagaaaatttccaacatttaaagtcttatctaatagattctaacttctctgaaattccaatttcagaaagattgagatttttattgacaaacttaataatgagctaatcaggaaatcatgaaaaaacgtaatttatttagaaaaaaatttaaaaataatgctccgcgtaatgagcgtatcgatgtaagcatcattatcccaatgtataatgtagaaaatttaattggtaaaactattgaatgcttaaaaaataatttctgtaaaatggaagtattgcttattgacgatggttcaaaagataacactgttaataatgcaaaattagccgttgctaacgataaaagatttactattcttcgaaaagaaaattctggtgtttcctctactagaaactttggaataaaaaaaggaaaaggagaattcatttttttctgtgattctgatgacattttagaaggaaatgctattgataaactactcattgcagcatttgctgaaaatgcggattatatttacggtggcatcaaaaaatttaataaagaaaaagagtggactattcctgttcacgacaaaaataatctattcctacaaggaacaaagaccatcgataagaatacagaactatttttatcaatgtctcctggggcaaaattaatacatcggtcactgttaaagaataaattttttccagaagatattcattgtgtctattgtatcggtaactatatctacaaatatacagaagatcaggtcatattcttcaatatatttttagatgctaaaaaaatctattgtatcggtaactatatctacaaatatagagagcgagatttagaaaacaatgaacgatcaattacacagcaacgtgatataaaagcctttgcattttttattgatattttgtcagttgttgaaatcaatagaaaagttttggaagaatctgctttaactgactctcaaaaaaaacttgttcttaaagcatattttgaaagagccttaacctttgatgtatggcctctatttctacgtgtcttaaagtttgacaataaaaaatctagcgaggcgataactctgataatcaatctaattaatagtgtagataaagagtttttgaattacactccaggatttagatattttttcttaagagttttaattgataatattgattatattagcatcaaagattatttactatacaaatcactagtagctctaatcatttcaaacttacaagaaaaaactatagaggtttgtgaaaattcgccaaactggggaaatagattgacagaaaataaaagtattatagctaaaaatggattcttaaactttttattattacgtcagaagaaaaaactttctagatttttaaatagaaatagagaatttattggtaaaaatttatttttccctttaatgaaacttattcctatcaataagaataagattgtgtttgctacatcatcaaaaggaaaagcatctagcaatttcacatatctgctaaatgaactaaaaaaagataacaagaattatgagataaaaaaattccttggtttatcaaatgaactaaaaagaaacttataccgctattatcatttagccactgcaggaacaatttttttagaaagctattatagccccctatataatgtaaaattaagaaaaagtactaaagtagttcaattatggcatgcttgtgccggctttaaaaaattcgcatatagctcactaggtcaaggcgatgctaactccgaagaatttgagtatcatgctcataagttttatacacatgttatgacaagctcagatgataccagtattatatattctgaagcattcaacttacctttagagaaaatgcatagtttaggtgttcctagaactgatttcttcttcaatagaagaaaaatgaattctgtacgaaatgaaattttagaaaaataccctgaatctaagaatactaaaaatgtactttattctccaacatttagaggaaatcctagagagcgtaaaaacttcaaattaccatttgattttaaacaatttgataaattaccttatgaatataagataattatcaaattgcacccagcagtcgatatttcaagtataaaaataccatatcattatagaaatcgttttttattattagatagttccgaagatgttaaccaatggttatgctttagtgatatattaattacggattattcttcattaatttttgaatatgcattactagataacaattatttatatgcgtatgatatcgatgagtacttcgatgaacgtggtttctataatccatatgaaacttatgcatatggagaaatagtcacaaataagagaaggcttattgatgctattttaactactgaaaataacatggatgattatgctcagaaaaaagaagcttttaaagaaaaatttatgtctggctgtgatggcaaatcatctataaaaattctatcttatattatgaaataaatttgaggcttatatgaggaacataaaaacgttattagaactcataaaaaatgaagataaaaacttttctttttataaatcattttacaataaatctatctattatgattttccattctttcatcacaaagagaataaatgggataattttaccgtcagcattgatttagtaaaacaaagattttatttttatctacgctctcacttaaaatcaaaatcgtatcgtctgtttagctacatgaatgggaaatactattgttatcacatcgctaaattaactgacctagatgtgtcaggttatatatttacttacaaaatgatcgaaaatttatctatttataacacacctaacatagagttagaaaaataatacattatatatttacttatactaattaaaagcaccaaaaaatttccacttaaaagttaattgagttatttattaaaaataaaattttaatataaaacatcatatataattattttttagctatctttctattcacttgaaagatagatcgcaaacttatcatttacataaaggaatattaaatttgcagaataaatctaatatattgcggggaaagagcagataaaaccatgtcatctgccctcattttttcatacggaattaaggaaatccctcacctagcctcattttttcctaatgaagaa

SEQ ID NO: 28 - DNA, 600 bp, Haemophilus influenzae
ttgacgatggttcaaaagataacactgttaataatgcaaaattagccgttgctaacgataaaagatttactattcttcgaaaagaaaattctggtgtttcctctactagaaactttggaataaaaaaaggaaaaggagaattcatttttttctgtgattctgatgacattttagaaggaaatgctattgataaactactcattgcagcatttgctgaaaatgcggattatatttacggtggcatcaaaaaatttaataaagaaaaagagtggactattcctgttcacgacaaaaataatctattcctacaaggaacaaagaccatcgataagaatacagaactatttttatcaatgtctcctggggcaaaattaatacatcggtcactgttaaagaataaattttttccagaagatattcattgtgtctattgtatcggtaactatatctacaaatatacagaagatcaggtcatattcttcaatatatttttagatgctaaaaaaatctattgtatcggtaactatatctacaaatatagagagcgagatttagaaaacaatgaacgatcaattacacagcaacgtgatataaaagcct

SEQ ID NO: 29 - DNA, 282 bp, Haemophilus influenzae
aattattaaactttaaaaataatatatttattatctgtgtagatgatggttcagtagatgcagctaagattattaaaaaatggcagcgtaaatatcctaaaaatatcacatatatctataaagaaaatggtgggcaagcctctgcaagaaatgttggaattgagcacgttcaaaccgaatgggttactttcattgaccctgatgattttgttagtaaaaactatttttctgaagtcgataaacaaatatcagagaatgttcactaatagcttgcctctgttt

SEQ ID NO: 30 - DNA, 645 bp, Haemophilus influenzae
gcaaatcatagtgacaaagaactgactttactagactaccaagaaagcttaaaaactgtagatgtagctgtattattagttaaccataaagaatttgttgaaaataaaccgcactttggaaacaatgttgtaatcgttgatacgaaagggatttggtaattttcactattataggaaaaaactaagtactggtagaattaatttatctaccagtactttatgtatataattaggtaaaacagatgaagattttaatttttggtagctatgttacaagagatgcttttacttttgataaatcaaatgaatttgaattagatcgatatttcgttagaacatctctagcaactgcttttaattcagagccaatagaagataattatacgatgcaattaacctctgcatttcagaaaaaaattgtaaatgctgagttaaaaaaagaattcaaagaactctttcttcttgggaagtatgattaccttgtgattgattttattgatgaccgatacaacctgtttaaatttaagaatggttgtaaaactcttcttagtgctgaattaaaaaatactgatttcttagataaaaatcaacataacggagaaataatttctggattctctgaagaatggtttgaagagtggaaaa

SEQ ID NO: 31 - DNA, 1369 bp, Haemophilus influenzae
gttgaaaacaaaccgcactttggtaacgaatgtagtggtagttagatatgaaagggatctggtaattaaagataagggggcttttgtcccctaaataataggaataggtatgtctaaaagaaaaaaaatcaaaaaaaatcaaaaaaaatatcactcttatgaagaaaaaatagtaccgcagcaagaattttttcagtttaatacaagcttaacgctgtttcacaatacagctatgaacaagataacgaatccaaagttacaaaataaaattgatttcttacaggaagaaaagaaaaatttaactaaagcgatccgtgaatttgaagtcgtccatagagaatggttacaagaaaaatctaacttaactagacagaaagaacaattagaaaaaaaagctcttagttatgagcactcaatgtcatttcgattgggatacgccctcatttttggttttaaatcatggacaggctttaaacatttaattaaaacactttacacactaccttttgagaagagaactaaaaaacaacaacgtacagaaatatcagttacacttcgcaaacctgattatttaactattaattggaaaggtcgccgtactattactcaatggacaagtctacctcaaaaagagtttaaggattttattgagcttttcgcagtggagcggcttgaattaagaaacagagggttctttcaaactattgcttgcaaaaatattgaccaacttgtcttatcaatcaacattgatagtgaagaaacccctaagcagactaaacaagcactggcagctattacattcttagataaagataataatacactagaatctagtattgaattaccacaaagtaaaaaactggggaaatactatttctaccttaatacagagaaagatcaccagggtaatttatttattatcccacctttagattgtgaaaatataaaattagatattgttccttgggatataaaaggaaaaatatctgtacataataaagtaggcatatctcactatacaaatggtattagtatcattcttccaacatataaaggagttaatactattaagaaatgcttagattctttaaataatcaagacctaagttatagcctatttgaaattattgttgtgatgaatggtcctaaagatggcacagaagagttattaagacaatataagttggaaaatcctaatcttaatttaagatattattctttgaaagaagctaatgtaagcaaagctcgaaatttcgctattcagaaagccaaattttcttggactacatttattgatgatgatgattatgtagacacaaaattctagacttatacagtaaagctatgtataacaacattaacattaact

SEQ ID NO: 32 - DNA, 700 bp, Haemophilus influenzae
aaaagctcttagttatgagcactcaatgtcatttcgattgggatacgccctcatttttggttttaaatcatggacaggctttaaacatttaattaaaacactttacacactaccttttgagaagagaactaaaaaacaacaacgtacagaaatatcagttacacttcgcaaacctgattatttaactattaattggaaaggtcgccgtactattactcaatggacaagtctacctcaaaaagagtttaaggattttattgagcttttcgcagtggagcggcttgaattaagaaacagagggttctttcaaactattgcttgcaaaaatattgaccaacttgtcttatcaatcaacattgatagtgaagaaacccctaagcagactaaacaagcactggcagctattacattcttagataaagataataatacactagaatctagtattgaattaccacaaagtaaaaaactggggaaatactatttctaccttaatacagagaaagatcaccagggtaatttatttattatcccacctttagattgtgaaaatataaaattagatattgttccttgggatataaaaggaaaaatatctgtacataataaagtaggcatatctcactatacaaatggtattagtatcattcttccaacatataaaggagttaatactattaagaaatgcttagattcttta

SEQ ID NO: 33 - DNA, 19181 bp, Haemophilus influenzae
actcatcttcacgccctgcaccagtatcattataacgaggatcgataataatcattttggcattggaacgagctttggcttgttcaatacaataagttaaaccacctccactcatacgagtttctgcaggattattaccgaataacactattaatttggtgttttcaatgtcagccattccatttcccaatgcccaaccaccaccataggtataatctaaaccgactgcaatttgtgcggtgctgtaatcaccataatggtttaaatatccaccaatacaattcataaaacgagcgatcatagttgatgcaggtggccaagatttagccattgtgccaccgagtgttcccgtaccatagtttaaataaattgattcattgccatattttttgatattgcgtttcaatgcgtctgcaatttcagttaaagcctcatcccaactgattcgtttgaatttaccttctccgcgtttacctatacgtttcattggatattttaaacggtctgggttatacactcgacgacgcatagaacgtccacgtagacaagaacgaacctgatgatcaagattatatgtttctgtccccgtattatcggtttccacataagtgattcggttatcttttacgtgcattcgtaatggacaacggctaccacagtttactgtacaagcactccaaacaatacgttcctgactattttcattgaggtattgtgtctctttagccattacattgaaaggtaaagtaagatttgaaactgccagtgcagctcccgcagatgacgccttgacaaaatctcggcgacttatttgattaaagttactcataatatcgtcccccacagtaagatcgagtaataactaaaacgagttaattataatctttgtatagaagttttacttgagccaaatcaactaattattaataggtatattatttatagggtattttagtaggttgatgtttttataaattttggaataacaaattgactatagatttaaaaatccgttagcatagtggcgtattagtttatctaatatgttaagtttaactattattaactagaggatcttttcatgaagaaacttatcgcggttgcggtgttttctgcgtgtggctcgttagctcatgcaaacaccattattccaaactacaatacagacgcccatctttacgaattcacgcaaacctacgatttagttgcaccaaaaggctcgcaaggacaaaccaatttatgggttccattgccatttaatggagaataccaacaagtgaaatcgattcactttgaaggtaattacatgaatgcctatgtaacagaaaacaataaatacggtgcgaaaaccttatttgccacttgggataaagatgcacaaaaacgtgatttaaaagtcacgatggtcattgaaacaaaagaccgtgaacctatggtgaaaggtgctttagagaattatactccgccaaaagatattcagtattccgtggatgtacaagagtatttaaaagctactcaacacattaaaactgatggcattgtgaaagaatttgctgacaaaatcgtaggtaaagaaactaatccattgaaaaaagcagaacttattcaccattggatcgtaaaaaatatggaacgtgataattctgtattaggttgtggtgacggcgatgtagaaaaaattcttactactggcgtattaaaaggtaaatgtaccgatattaactctgtatttgtggcgcttgctcgtgcggcagacatcccagcccgtgaaatttttggtattcgcttaggtgcggcagagaaaatgggcaaatattccaaaggtgctttcggtagtgcgaatgaacaaggcatcgcaaacgtaagtggcggtcagcactgccgtgctgaattctaccttgcaggatttggatgggtaccagttgattccgcagacgttgccaaaatgcgtttagcagagaaaaaatctgttgaagataaagatacacaagccgtaacaaaatatttgtttggtaactgggaagcaaactgggtgggatttaatcatgcccgtgacttcgatttatatccacaaccagaactggctccaatcaataacttcggctatccgtatgctgaagtgggtggcgatccgttaaattcctttgatccaaaagaatttaaatatgactacgtctctaaaaagctctaataaatccttttgggttgccatagccaccgcacttagtgccgcggtggcatcaactttgtgctgcatcgcaccattaatctatttagtgttcggcgtgtcatctacatggttgattggcttaggcgaatatgattatttgcgtattcctatgcttatcgtttcattatgtgccttcgcctatggattttggctgttgatgttttccaaaaaaatcatttgtagcaaatatatttcccgtaaaaaactcatcgttttatattggattgtatttatcgtgatgatttttttcttaacctatccaacaattttgccttggattttagaattagctaattaggaataaaaatgaagaaattatgtaccgcacttttgctttcgctgtttgcaatttctttcgctcatgcgaatgaaaccaaacaaattgtgctaaaagtgaatgaaatgaactgccagctttgtgcttatttagtgaataaagagttacgcaatattgatggcgtgatttccactaaagcctccattaaagatagaaccgttactgttgtagaagaccccaaagtggctgatgagcaattgattaatgccattcacaaattggaatatacaacggaagtgattaaataaagtgataaaaaaataaaagtgcggtcgatttttgagaggttttaaaactctttagaaattgacctcactttttatctttaagaatcgacttatttaatcatgccacatgccatacgtgggccgccaccgccaagtggagcaggatgatctgagtggttatcgccacctgcgtgaatcataatagagtgaccacgaacttcatctaattttttaagacgtggagctaaaacagggttagttgctgtaccatcatgtaatactgttaaagccggtaagtcacctaagtgagcatcatcttgccatgggtaaccatgttgttttgcacctttagaatcccagtgaccgccagtcgctaaacctgctattaatttaccgtctttttcttttggttcacagcttgggttttcataaatatggaaaccatgtaaaccttcagctaaaccttgtagatttggtgtaaacactaaaccataatttgattcagtaatggttacagtccctacatctttgttaccatttgcaggatctgattgttgtacttttacttcgatggaaggatgtgctggttttgccatatggtcatgcgcattttccacgccagcagcacaaattccgctgattgctaatgctacgagagttttcattttcatcataagcttctttactttagaaagactcaaattgattctacctaaatatcaataaaaaattatctttatattaaagacttaacaattaaaccaattttagctttctatcaattttaaattaagacattacttattaattatttttgagtttgatcataatattgatacgctttgtccatatcttcaaaatggtgcatcataccattttccaacaccacagctttatcacaataagatttcattgcacttggactatgtgaaaccaaaataatggaacgatctttacgcttttcaaataattcatatttgcatttctctgcaaaccgagaatctcccaccgctattacttcatcgatcaaataacaatcaaattcaacagaaagagaaagagcgaatgcaagccgagctttcatccctgaagagtatttcttcactggttcataaagataatcacctaattcagaaaattcttttgtaaaacgagtcacatattcaggatcaacatcatataaacggcagataaaacgcaggttatccatcccggttaagcttccctgaaatgcaccactaaaagctaatggccaagaaatactcattgaacgatcaatagtgccgcttgttgggggttctactccactgatcaaccgaataagtgttgatttgcctgctccatttcgtcctaggatcccaattttctcacctttttttaactcaaaattgatatcttttagtaccgttttccaaccactattagtgtgatactttttacacactttattcatacgaatcattgcgcctcaatccctttactgaaatttttaaccatcactaagccaattagcaataaagccaaatcacttacgactaaaaatccgatgctctcataagtgactaccgtatccccaaagtaaccatgacgaaacatttccgtaccatgaatcattggaagccaaagtgcaatagattgagcctgtgatggaagattatgtacaaagaaaaatgctcctgaaataggtaacaatacgaaacttaaggtcccccaaattttaccaaaaacctcaaattgctgagcgatagcacaaataattaggcctaagctaaaagcaaacatagccatgagaaaccatgcaataagcatataaaacacatcttgtggcgcatcaatccaaccgatcatcaccaggattaccataaaaagaatttgagcaatagaagcccccgccacctccagtaaaactcgtgtaaaaatagtatctaacacacgaacatttcgatgataaagtaagctaagatttgctgaaattgatccaattgcacgattcgaagcattgcgccacatcatcgccataggatagcccgtcatgacaaaagcaatcatatttaaagttgaaaatttatcagcacgaataaatttccacatcatcacaataaaaaaagtcatgagtaaaggctcaacaaacagccatagaaagccaatatttttacgcccataacgagagactatttcacgcataagtaatgcgttaatcaccctcccttgaatcgctaatgattgtttaaacgttgtttgatcaccatattgcattagtttttatgctctcttatgcttgcgataagtaaactcaatacaccatataacattaaaccgacaataaaggtcgcaatgatattataaatacgacttggctctaatgcccagtcaggtttacttggttgactaattatttctaaataaagttgttggcgatcggcttctccgcgagtgttttgtaaagatgtcattgctgcggtcaattgttgttgagcaagctcattgtctaagactaaacgttgataatctgcagtttgtgttgcagctgaatttccattacctgataattgacgtgtttgttcatcaatttctcttttcaaacttttctggcgcatttgcaatgcgggtacttgtgggttatctggcgtgattgaaattaattgtgctaattgagtttcaactcggattaattcacttttcaacgttgaaattaatgaaatttgcacgccagattgagccggtaaatcaaaaattttatttttaatacgatattgactcaatgcttgtgcactttcattcacatttttctctgcatcttttacggcctgctcagcaaattcaatcgtatcttttcttgcacgctcatttaatctattgattaatgattcaccttctttcaataagcgctcattgatttgataaccttcttcagcatcaaaagcatgcactcgcaatgttgcaatgccagaaatagaatccacatcaacactcaaacgttctttaaaataacgatagaatgcctcttgtgtatcattcaaaccaaacccattaaagcggcttaataaatcgcctttttcagaataaaaagtgcgaagaggcaaactttgctctaatgctgatastgctgtacgtgaacgcatatactcttgcacagagtaagtatcatcctgtgaacgagaaaaacccgtactttgcaataatgcaccaactccgcttaaagaagactgactgcgaggagaacgtactacaaaactagattctgaaacatagatatcagatgctaatagaccaaaatatactgcagaaaaagcagttggaatcaatacagttaatccaaaaagcgaattcattttattccaaagtgatttcttctttttcttcggtgccactgggttttcggttgtcatattatccccttaaattaataacttctgatcgtattggtcgtactcgttaccggcgaggtaatcgagaaaatcattctcaagaatttttggaattctgaaagcggtgcattcgacacatatacaatatctttatcttggatagggaagcgttgtaataaaaacatggtttccggttgtaataaattcgcatgatatacggttggcacttccatgccattaccataccccttagccgcccattcttgctgtgctttagagtctaattgtgcaaacggcatataacggaatacaaagacacctcgtgggtcagaacgcgtgtcaatcaaaccgcccattttaccaatcgcttcagcaagcgtaataccactgctagaaaaacgtaactgttggttattacctacagcgcctagccctgtaaatttatatggcgtatttaaaagtgaaaccacatcgcctgcacgtaatacaatattttgcttaggatcggcaattaaggtttcaaatgccaatgttttcacttgatttcctcgcgtaagctgaacagtcacatcttcaatattttctgttgatccacctacagcagccactgcatctaaaacacgttcatcatttgcagttaatggcatacgcacagtactaccttgacgaataacaatcacatctgatgaattattgttagcaattttaactactgcctgtggttgatttgctttacgtctaaggcttgccacaatttgagcttgaataacctctggtgtcttacctgcgacactgatatttccaacaaatggcacggttaccgtaccatttttatttacgatttgtgaaggtaattgtgtaacatggccactgccctgtccttcactactaaatgttgtaccaaataacactgctggaggggcttcccaaatggagatctctaatacatcacccacatttacagcaccagcataaccgctgctgccagacacatctgcaaaccctgaaaagcgttgactttgttgtgccatgtacatttgttgcacgagtaattcatttaactcaactacattaacttttgctgctaactcagatgcatcagctccttggtttatatctaataccgctgaatgacttggtcctgaagttggtaacgaagaacaagcggaaagtccaaacatcaatgcaaagctgaccgcttttagtggataatttttcattgaatatctcatctcatgattaggaataaaaacgtcaagacttgcaaaatattgcataaatcattttcgctcatatgcgtgaaaataatcctgcagagtatagcatttattcaatcaggaatcaaaatttttatcccctctttaatattaatacgatttatgtattgtttatatttttataaatctcataataatttaaaattacattcatttacttttttgataattgacaattcatactaaataggctaaaatcaccagttgaactagccgctatacaaacagcaagaaaagctaataatgattctaaaatatatattagcgcttatatgctaaatataattagtaaggttatattaatatgaaaaaactaaaaaaatttgttacgaagccacacatattttttcgtgacgcattgaataataaataccctattataaataatgaacaaggaattaaagaattagatgaacgagctgttctctctcatcaagaaaatctggaaaaactagaaagctcgctaatgaatacaccaatacctattgatgtcgttttcacatgggttaatgataaagatgaaaaatggcaggaaaagaaacagcactattcgaaactagcaaacaactatgcattctatgcgaaagataatgtcagatttgaagaacataacgagctattttactctgtcaaaagtgtacaaaaattcttaccttgggtacggtacatttttatcgtaacggataatcaaataccacattggcttaataatgaagattcccaaattaaaattgtggatcatcgtgagatcattgatcacgattatcttcctacgtttaattcacatgttattgaggctaatctacataaaattccaaacctaagtgaacattttatttattttaatgatgatgtatttgtagctaaacctttaaaaaaaagccatttttttaaacctaatgggctagcctctattttcctatccattaaaaacttagataaaatgtatgcaaaaggtacaactacgcctacattactcgcttcaatgaattcaagacgcttattacgaaaaatgtatggacaagaattaaacatacaaacaccactaattcactcctatattccactcaagaaaagtgtttttgaaaaaatatggagtgtgtttaaagaagagattgaaagttttttatcaaatagatttaggggtaaaaacgatcttaatctcgctactttttttgtaccttatgccatgtatttagaagggaaaagtgttcttacgcctgaaatttgctattactttaatattcgctccgcaaatgctaaggcacagtataaaaagttattacaaaaaaaagagaatggaaatagaccacattctttttgtattaatgatacttctagttctaataaccatttttatcataaaaattttaacactttcataaatctatatttttaggaggatacatgaaaaccaatttcatattcagtataatcatgccaatttataatgttgaccaatggttagaagaagcaatattaagtataataaatcaaaagaaaattaattttgaagaaaatgttcaattaattttagttaatgattgtagccctgataacagtgaagaaatatgtctaaaatttagaaaaaaatatcctaataatattttatattacaagaatgagaagaacctaggattatctggaacaagaaataaaggattaacccttgcagaagggaaatacattaatttctttgatcctgatgatacattatctccatctgtactttatgaagtaaataaattttttacacaaaattcttctcaaaatttagctcatatatcaatacctttagtattttttgaggctgcttctggattacatcctaaatatagattacttggaaataaaaacagaattattgatttagataaagaacaacataactttatactttcatctgcaagttcattctatccaagagataatattaagaaaaataaatttgatacctcattatttggagaagaagacaccctatttaattttaatatctacagtaatattaataaatttggctatgtgtgcgaaaatggcgttcaatacaactatagaagaagacaagaaggaggatcacaagtagacctaagtagagtaaaaccacaagcttttattaccccaatccaaatattagaaaatgttaatgcaaaagatcaagttttattctatgaactaatagcttaccagcttagatcaagaataaaaaatataaaacctgaaatattccagaataagaatgattataatcatataattaatagatatagagattttatgtctctaattcctaaagattttattttacataaaacaaagtatttagaaacacaagaacaaaaaattgactttatttcagaaatatatagaaagaatctaacaatagatgaagatgcttatattaacatagatgattgtaaaatatttaaatgcaatgatttaccattggatataaaaaatattagtattgaaaaaaatgttcttataattgaaacattatttaataattttaacatcgaagatcttagcattgttataatggacaaaggtaaaaatattataaaaccaattaaagaatattattgcgacagcctctatattcataaatgctgtgatataaaatcaagtaataacatattatattctagattcgaaataccagtatttagaaaaggtgaatatcgactttattttaagagaaaaagcaatggcttcttacatatagtaaatagacttagaacatatagcgaaagtccattccttggcaatggcgtatttaattcaaatttatttaagctttattctgaacagaatactagtatatctttatataaaaaagctttttatataaaaagctctactgtaattaataaaatatcaaataggataaaatcattatttttaataagaaaaagacataaaacatggaaatggttaagattattaaaacttaataaacctaagtactggctatttaatgaccgaccaattaatgctaatgataatgcagaagcattctttacgtatattaataaatcggttcctcatatcgctaagaactcttattttgttctagataaaaattccccagatataagtagaatcaagaaaataggtaaagttatcattcaaaatagcctaaaacataagttactttatttaaattctaagtatatatttacatcgcatttagcaacatcattttttaaaccaatatcatttaagcatttaaaatattataatgatttgatagaaacaaaaattatatggttacagcatggtataactatgaataacatagaaatagctgcaaataaatttaacaagcatatttataaaattgtaacggcagcaaactttgaaaattcaatatttaaaaataagaatttcttttttaataaagaagacttatttaatgtgggttttccaagatatgataaattaattaagaaaaaagatgaagataagattgtactcattatgcccacatggagatcctatctaagtggaaatattctaaaaaatggattgcatgctgaattagaaatctttaaagaatctgactactataaaaattttgtcgatttattatcaaataaattactaattaatacattaaaagaaaataatgttattattaaatttgttttacatcctggatttaaacagtatgctaaatactttaaacagttagaatcaaatgaaattcttattatcgatgaattatctctatcttacaaggatctttttaatgaagcatcattattaataacggattattctagtgtgttttttgatttctcttacaaagaaaaaccatctatatttttccaatttgatgaagatgaattctattcaaaacactataaaaaaggattctttgactttactagcatggcgccaggaaaagtaacttataacacagatgatttaatatctgaaattattaaaagtataatttctaatttttctattaaaaatgaatatctatatagaattagaaatatgtataaatataatgataataaaaactgtgaaagattattaaatgaggtattgaaaaatgaataataagagacacattagactaaaagaattttttccgggaatccatagagttcatacaccatcagaaagttatataaaaaaatcccccttactacaaaaaaatagatcttattttcagaaagttgacaataatggatttatgcaatctcatttaaatatccaagaagataaaaaaatttttttagttggtgattcattcatagaaagtatttttatcgatgaatcaaagagaataaacgcaattatggaagaaagctttctatttcaagaaggaaaagaagtaaaagtatataatgctggagtatctggttctacagggttaaacttatttaaccttatacttaataaaattattattttaaagcctgacgttattatatactctcaaccttcctgtgatttttctgctttactatatgaaaatggatattataacaatagtaaatatttttctaatattattccatcagtagaaagtgatgtttttagattcaaaactattcaagataatttaattcagatacagaataatattattatgctatctaaactatgtgagttatacagtatcgatctctttatatcaacttgctgttcaaattcatctaaacgccaacttaaaatgatgaatgatattattagagagaattcttatcttgggtataaggttattgatttggacttgatagtaccaaaaacagaggcctatttttatgataaacaacacttaaatgaatatggtagtaatttagttgctaatatttatctatataacgttagaaatttttcagatgacatgccaaaaaaaacaatacaaaaacatcatatacaaaaaataaatggcgtctttgaaataacctcaaacaacttagaaaaagagagcaactctattttgttaaaaataaaaaacaatgaaaaacaaaatcaagattttgaaataaaaataacatatttcaatgaaggagaaataataaagaatgatgtaaaaaaaatattattgttaccattgcatagtatagaatgctcatattttatagaagaactacatcagactaaagtacttatagaaccaatttctatatcaaaaaatattgaaatagaaatcataaaatatactattactttattatgttagctaaaagtatgatatgtttttttaggcattaaaaacacaatcaatcatataaaaattcaaattagaaccatgccaactgctctgattttctcgcacggaatcaaaaaaattccgcatttagcctcatttttccctaatcaaaaattggctttcgcgcgcattttttctgtgccgaaggctgattctgtgttgggttgggggttacgtccttccacgaaaaaatcacgtgcatatgcacaaaagcataacttaccgtttatcgccttagaagacggttttttacgttcgctaggcttgggcgttgatggttatccaccactttctatgatcgtggataaattaggtatttattacgacacgactcgtccctcaacattggaacagcttgttctggcgggagaatgtgatgaggttttagcggaaaaggttcgatcacagattatgacgcatcaactttcaaaatataaccaaacattagtggattatgagaaagaaggtgacgagccattagtgcttgttatcgaccaaacctttggcgatatggcggtgaaatatggtcaagccgatgctgagcattttgcacagatgttacaagctgccattacagaaaatcccaaagccaaaattttagtgaaaacccatcctgatgtattaagtggtaaaaaacagggctatttttcgccaaatgaaaattatccaagcaacgtgcatttttttagtgatcccgtcaatccgatttcgctgattaaagccgtagagaaagtttattgtgttacctctcaaatggggtttgaagcattactggcgggtaaaccggtggtgacgtttggcgtaccatggtttgctggctggggcgtgacggatgatcgtcatcagaatgctaaagcattggcacaaagtgagcgtcgtaaagtgcggtcagttttacaactgttttatgcggcatattttcaatatactcgttatttgaatccaaacaccggcacagctggaacaatttttgatgtcattaaccacatcattcacaccaaagcacttaacttgtgcttacaaggtaatctctattgcgttggaatgtcgttgtggaaacgtgcggttattaaaccgtttttccgtttaccaagctgcaagctacattttgttaaagatgtgagtaaactcaatggtaaaacattcacaaaaaatgaccgcttgttactatggggaaccggtaaagaagctgcgctaaattatgctaaagcgcataatattaacatgctcagaatggaagatgggtttattcgttcagttgggcttggctcaaacttggttgcgccactttctttagtgtgtgacgatttaggtatctatttcaatgcagaaagtccttctcgcttagaagagattttgcagcatcaacaatttaccgaagcagatttaatcgaagccagtcaactacaacatgcgttggtatcgcaacatatcggtaaatataatgtcggtgaaggaacattttctttaccgaatacagtcaaaaagaaaattttagtggtaggacaggtcgaagacgatgcctctattcgaactgcctcaccgaatattcgcactaatttagatttattaaaaacagtatgcgccgcaaatcctcaagcttatatcgtttataagccacacccggatgttgtaagtggtaaccgcattggtcatattgcagaatcgcaagcggtcaaattagcggatcaaattgcaaaaacagaaaacgtgcttgactgcatcaatgccgtggatgaactacacaccatgacatctcttgcaggttttgaagccttgctaagaaacaaaacggtgcattgttatggtttgccattttatagccactggggattgaccgttgatcactgctcttttaaccgccgcaatcgaaaactttcattgttagaacttatttctggcacattggtctattatccacaatatgttaatccgaatacgggtgagctaaccaatgctcaaacagcgattgaaattttgaaagcacagcgtttagcgcttaatcacagcggtattcatcgccattggctagctaaacaatatggcaaattgcagcatttagtgtgcgcattgcgtacttaacaacgccagttatagcaacatatcttaatcatataataaataatcgttttcgtatgaaccactatttagatgaattagtccaaagttcacagcggatcttgcttttgcaagggccgataggtcacttttttgctgatctttctgattggttagtggggcaaggaaaaaccgtttataaaatcaattttaacggtggcgatgagcatttttatccattatcgattgaaaatacgattgcttatcgtggctcagtgagtgagttctacccttacctgcaactattttgcaaacaacataatatcgatgcgatggtttgctttggtgataaccgtaaataccacaaaatcgcaaaaaaaatctcacaagatagacagattctattttgggtgttggaagaaggctatttccgtcctgattatgtaacactcgaaaaactaggggtaaatgcgttctctcctcttcctcgccaggcagacttctatcttgaacaagccgaaaatttacctgagccaaccgtaccacttaaattagccaaaggttttttaacgatggcaaaagtagcaatagcatattacgttagtgcatatctagctagagataattatcctaaatatcaacaccatcgtattattaaccttaaatattacatcaaacattggatcatttcgggcattaaacggacttgttattatttgcgtgatcgcatgtttgcaaaaaaagtaacaaaaggagaactcggtcatttttttattgtaccgctgcaagtttatgatgatagtcaagtgaaaatacattgcgacttcaatagtgtcggtgaattcttagctgaagtactacggtcttttgctacatctgcgcctaatcatttaaacttggtcattaagcaccatccaatggatagaggttttgttcattatgagtctgttattctagagtttgaaagctgctatcctcatttaaaaggaagattattttatgtacatgatgtgccattgcccgttctacttcgccgtggaaaaggcatggtaacattaaacagtacgagtggtatttcagccttattacatggaatgcccgttattacgcttgggcgtgcaaattatcattttgaaggtctcacacaccaaggtgatttagcttcattctggcaaaatccaaccaagcctgatatgtcagtatttgaggcttaccgcaaatatcatctcaataaaaccatgattaacggcagtttttatactcgagtcatccttcccttacatcaggatgaaaattaatcaaacctatccgccaataaaaactgcaagacagcatccattcttaaatgtgggatgctttcaccttgttgtaaaacttgaggttcaaaggaatcaaagtcaaaagattgttttgaccagaactccgcattaggtaatttacttggtacagatcctggataaactgtaataagttgcttatctaaagaacgtactccttgaatagctttgatttgtttaccgttttgattaaccaaaacctgttttatcgctcgaatcgcagcaattgccgtatattctgtatcgatgccttcaaattctacatgacggccgccttcttgcaccaattggcgcattaggctaattaaattttgaatttgatctgttgtaatatgatcggcttttgttgcgacaaacatgagtttatcaatattcggagaaaacaagcgattaagtaaattacgtttcccataatggaaatttttgaaaagctcattaagacctgtttgcatatcaataaaggcttgttggctatgattcagtggtgttaagcaatcagctaaaattacttggcgatcaaaagtcaaaaaataattttcataaaagcccttcactaccttattacgataataatcgtatcgcttattcaacaccgcaaaatagctattggatttcgcctctcttttcagcttttgccattgctcctcggacaaatgcaataacgggaaaaattgcaatgcaggtgcaccttctaattcgccagccaggcaagacaaatcggcctggttgaataaattgcattccttctgctttgcaagtgagcaaatagtcggtataagattttgcaatctctgctaaaacatcttcattgacgaccgcacttaggtcgagctttttcagtttatccaaccactcttgggcaaactgttggcgaatacccgatgtaattttagcttgttcaagtgaccattgttgaaaatctagattcagcaaaggtaaatccaatagccattcaccaggataatcgaaaatatccaagtataatgtgccacgttctttaaaatgacgaagtaagcccgattgacgttcaaaacgaatagcaaggcgtgtttcactcacccctcgagtagattggcaccattgtggtggattattcactaaatcattgagattcgcttcataatcaaaacgtggaatacttaaatcctgttgaggcactcgttttaccgccaaaatagactgatttcgggcagcctcaaataaaggaagatgagcattttcctcttgattaatatgtaacagttgattaatcaaactcgtaataaaagccgttttaccgcttcgacttaacccggttacagccaagcgcaaagtgcggtcaaatccacgattgattattttacaattcactcaatttatcacaaaacggtctttaataataaagctatgttacatcgaaatgtcactttttgttttttattgtgtgggttgagcctaattaatctggcacaagctgccccgcgcatacctaaaatgctgacagaaaatggcttaacatactgcacaaatgcatcgggtttttcctttaacccgcaaactgcagacgcaggtaccagtatgaatgtggtgacggagcaaatttataacaaattgtttgatatgaaagaccacagtgcagcacttgttcctgtgttagcacaatcctattcaatttcatctgatggcaaacaaattctcattaatcttcgtcaaggggtgaaattccatcgtacgccttggtttacctcaacacgtgagtttaatgcagaagatgtggtcttttcgattaatcgtgttttagggcatgatacttatctaccgacactttcagacgatgtagtaacctataaaaatccacaatatagaatctttcacgagcaagccaagaaggttcactttccctattttgaaagtattaagctgaatcaaaaaattaaaagtatcacagcaacgaatccttatcaggtcaaaattgaactgtttgaacctgatgcctcaattctgtcgcatcttgccagtcaatactctattattttctcgcaagaatatgcataccaattaagtgctgatgataacctctcccaattggatactcatcctgtcggcacggggccgtaccaagtaaaagactacgtttataatcaatatgttcgcttaatacgtaatgaggagtattggaaaaaagaagccaaaattaaaaatattattgtagatctttcagctgagcgcagtggtcgtttaataaaattctttaataatgaatgtcagattgcttcttctccagaaatcagccaacttggtttgttaagtgaaaaaaatgcctcttattatttacaatcaacagaagggatgaatttagcttatttagctttcaattttcaaaaatcgttaatgcaggataaaactattcgtcaagccatttcgcaaagcttaaaccgttttagaattgtacgaaacatttatcataacacggcaactgtagcgaataatattattcctgatatttcttgggcttcggcgattaacacacctgattttacttatgattatcagccttcaaaagcggaaaaaatcttacgagataagaaattagcattgaaaatgtgggtaataaacgaagagcaagtatataatcctgcccccattaaaatggctgaacttatcaaatgggatttagcaaaggttggagtggatgttaaagtgcggtcagtcacacgtacatttttaaccgaacaattacgcaatcacactgaagattacgatttgattttaacggggtggcttgcaggaaaccttgatcctgatggttttatgcgcccgattttaagttgtgatactcaaaatgaaatcacaaatttatcaaattggtgtaaccctgaatttgataagatgatggatcgtgcactgtcgactaatcatttatatgagcgttctaaagcttataatagcgcacaagaacttattttaaatgaattacctattgtgccaattgcaaacgttcaacgacttttagtcgcaagtggcacgtaaaaggcatagaaatgacaccatttggtagcatcaatttttctactttatattttatgaaaactaaggagaaaaaataatgctcctttcagcaattagacatatagt

SEQ ID NO: 34 - DNA, 740 bp, Haemophilus influenzae
ttattttaaagcctgacgttattatatactctcaaccttcctgtgatttttctgctttactatatgaaaatggatattataacaatagtaaatatttttctaatattattccatcagtagaaagtgatgtttttagattcaaaactattcaagataatttaattcagatacagaataatattattatgctatctaaactatgtgagttatacagtatcgatctctttatatcaacttgctgttcaaattcatctaaacgccaacttaaaatgatgaatgatattattagagagaattcttatcttgggtataaggttattgatttggacttgatagtaccaaaaacagaggcctatttttatgataaacaacacttaaatgaatatggtagtaatttagttgctaatatttatctatataacgttagaaatttttcagatgacatgccaaaaaaaacaatacaaaaacatcatatacaaaaaataaatggcgtctttgaaataacctcaaacaacttagaaaaagagagcaactctattttgttaaaaataaaaaacaatgaaaaacaaaatcaagattttgaaataaaaataacatatttcaatgaaggagaaataataaagaatgatgtaaaaaaaatattattgttaccattgcatagtatagaatgctcatattttatagaagaactacatcagactaaagtacttatagaaccaatttctatatcaaaaaatatt

(19) United States
(12) Patent Application Publication
(10) Pub. No.: US 2009/0286239 A1
(43) Pub. Date: Nov. 19, 2009

(54) Method of Detecting Individual Encapsulated Influenza Viruses, Primer Set for the Detection and Kit for the Detection

(75) Inventors: Mitsuko Seki, Tokyo (JP); Hirotaka Torigoe, Tokyo (JP)

Correspondence Address: BIRCH STEWART KOLASCH & BIRCH, PO BOX 747, FALLS CHURCH, VA, 22040-0747, US

(73) Assignee: NIHON UNIVERSITY, Tokyo (JP)

(21) Appl. No.: 12/226,436

(22) PCT Filed: Apr. 19, 2007
(86) PCT No.: PCT/JP2007/059004

§ 371 Date: Jun. 24, 2009

(30) Foreign Application Priority Data
Apr. 19, 2006 (JP) 2006-116104

(51) Int. Cl. C12Q 1/68; C07H 21/04

(52) U.S. Cl. 435/6; 536/24.33

(57) ABSTRACT

The present invention provides a method of rapidly, simply and accurately detecting capsular serotype Haemophilus influenzae other than Haemophilus influenzae Type b, a primer set for detecting the same, and a kit for detecting the same.

The method of detecting Haemophilus influenzae Types a, c, d, e and f of the present invention comprises: amplifying capsulation locus region II derived from each of Haemophilus influenzae Types a, c, d, e and f, using a LAMP primer set comprising one or more types of primers each having a nucleotide sequence that is identical to or complementary to a partial sequence in the nucleotide sequence region of the capsulation locus region II; and detecting the obtained amplification product.

### TECHNICAL FIELD

The present invention relates to a method of detecting capsular serotype Haemophilus influenzae. More specifically, the present invention relates to a method of detecting capsular serotype Haemophilus influenzae other than capsular serotype b (namely, capsular serotypes a, c, d, e and f).

### BACKGROUND ART

Haemophilus influenzae (hereinafter abbreviated as “H. influenzae” at times) is a causative bacterium of otitis media, pneumonia, meningitis, bacteremia, and the like. In recent years, appearance of various resistant bacteria has become a problem. H. influenzae is classified into a capsular serotype and a non-encapsulated type. Such capsular serotype is further classified into capsular serotypes a to f, depending on a difference in the type of a capsule.

Conventionally, as a method of typing H. influenzae, there has been a common method, which comprises selecting a cell strain recognized as H. influenzae by the combined use of a culture method and a biochemical method, and then applying a serological method such as a slide agglutination method to the thus selected cell strain to classify the H. influenzae into a certain capsular serotype. However, such a culture method and a biochemical method have required 3 days or more until infection has become clear. In addition, in order to precisely select H. influenzae based on a difference in the form or color of a colony, sophisticated techniques have been necessary. Moreover, the serological method has been problematic in that a noninfected state has been inaccurately diagnosed as positive due to a cross-reaction or autoagglutination, or in that an infected state has been inaccurately diagnosed as positive due to low detection sensitivity. Accordingly, only typing results with low accuracy could be obtained from this method, and thus there has been a risk of causing trouble with clinical diagnoses, the subsequent treatments, etc.

Under such circumstances, in recent years, a typing method utilizing a PCR method as a molecular biological means has been known (T. J. Falla et al., J. Clin. Microbiol., 1994 October, 32(10), pp. 2382-2386). However, in general, such a typing method utilizing the PCR method requires high cost, techniques and time, and it also requires special facilities such as a thermal cycler. Thus, this method cannot be easily carried out, for example, in examination rooms at hospitals, which may be poor in terms of human resources and facilities. Furthermore, in this method (T. J. Falla et al., J. Clin. Microbiol., 1994 October, 32(10), pp. 2382-2386), PCR must be carried out twice using three types of primers, and thus the method is complicated and is poor in terms of promptness.

As stated above, H. influenzae includes a non-encapsulated type and various capsular serotypes. Of these, non-encapsulated type H. influenzae is a contagion that causes serious diseases such as meningitis, epiglottitis, bacteremia and pneumonia particularly to children. In addition, there are nuanced differences in terms of pathogenicity and the severity of disease after the onset thereof among such capsular serotype H. influenzae. Therefore, not only for the original purpose of early detection of the onset of disease at clinical sites, but only for the purpose of confirming effects obtained by vaccination, it has been desired to develop a simple and highly sensitive method of typing capsular serotype H. influenzae. Further, from the viewpoint of the monitoring of H. influenzae infection, application of vaccine, etc., a method of simply and rapidly typing capsular serotype H. influenzae has been desired also in developing countries. Accordingly, it can be said that social need for such a typing method is extremely high.

By the way, as one of molecular biological methods other than the PCR method, a Loop-mediated isothermal amplification (LAMP) method has been known (K. Nagamine et al., Mol. Cell. Probes, 2002 June, 16(3), pp. 223-229; Nucleic Acid Research, 2000, Vol. 28, No. 12, e63). In the LAMP method, in order to effectively prevent an amplification reaction attended with occasionally occurring non-specific synthesis of a complementary strand and also in order to realize a highly efficient amplification mechanism, it is necessary to strictly design at least 4 types of (at maximum 6 types of) primers (a LAMP primer set) based on 6 regions (at maximum 8 regions) selected from target DNA.

### DISCLOSURE OF THE INVENTION

It is an object of the present invention to provide: a method of detecting capsular serotype H. influenzae other than H. influenzae Type b (namely, H. influenzae Types a, c, d, e and f); a primer set for detecting such H. influenzae Types; and a kit for detecting such H. influenzae Types.

The present inventors have conducted intensive studies directed towards achieving the aforementioned object. The inventors have focused on a LAMP method, which is more excellent than an amplification reaction by a PCR method in terms of specificity and which is also excellent in terms of promptness and simplicity. As a result, they have found that the aforementioned object can be achieved by designing and using a LAMP primer set capable of specifically typing various H. influenzae Types, thereby completing the present invention.

That is to say, the present invention is as follows:

(1A) A method of detecting H. influenzae Type a, which comprises: amplifying capsulation locus region II derived from H. influenzae Type a, using a LAMP primer set comprising one or more types of primers each having a nucleotide sequence that is identical to or complementary to a partial sequence in the nucleotide sequence region of the capsulation locus region II; and detecting the obtained amplification product.

The LAMP primer set may consist of an FIP primer, a BIP primer, an F3 primer and a B3 primer designed from the nucleotide sequence region as shown in SEQ ID NO: 27 in the capsulation locus region II, for example. Moreover, the aforementioned LAMP primer set may further comprise an LF primer and/or an LB primer as a loop primer(s).

The FIP primer may be designed from a region ranging from bp 3216 to 3288 in the nucleotide sequence as shown in SEQ ID NO: 27, for example.

The BIP primer may be designed from a region ranging from bp 3305 to 3387 in the nucleotide sequence as shown in SEQ ID NO: 27, for example.

The F3 primer may be designed from a region ranging from bp 3197 to 3214 in the nucleotide sequence as shown in SEQ ID NO: 27, for example.

The B3 primer may be designed from a region ranging from bp 3408 to 3429 in the nucleotide sequence as shown in SEQ ID NO: 27, for example.

The LF primer may be designed from a region ranging from bp 3239 to 3263 in the nucleotide sequence as shown in SEQ ID NO: 27, for example.

The LB primer may be designed from a region ranging from bp 3340 to 3364, or from bp 3339 to 3362 in the nucleotide sequence as shown in SEQ ID NO: 27, for example.

The LAMP primer set may be a combination of the nucleotide sequences described in the following (a), (b) or (c), for example:

(a) a combination of the nucleotide sequences as shown in SEQ ID NOS: 1, 2, 3 and 4;
(b) a combination of the nucleotide sequences as shown in SEQ ID NOS: 1, 2, 3, 4, 5 and 6; or
(c) a combination of the nucleotide sequences as shown in SEQ ID NOS: 1, 2, 3, 4, 5 and 7.
(2A) A LAMP primer set for detecting H. influenzae Type a, which comprises a combination of the nucleotide sequences described in the following (a), (b) or (c):
(a) a combination of the nucleotide sequences as shown in SEQ ID NOS: 1, 2, 3 and 4;
(b) a combination of the nucleotide sequences as shown in SEQ ID NOS: 1, 2, 3, 4, 5 and 6; or
(c) a combination of the nucleotide sequences as shown in SEQ ID NOS: 1, 2, 3, 4, 5 and 7.
(3A) A kit for detecting H. influenzae Type a, which comprises the LAMP primer set according to (2A) above.
(1B) A method of detecting H. influenzae Type c, which comprises: amplifying capsulation locus region II derived from H. influenzae Type c, using a LAMP primer set comprising one or more types of primers each having a nucleotide sequence that is identical to or complementary to a partial sequence in the nucleotide sequence region of the capsulation locus region II; and detecting the obtained amplification product.

The LAMP primer set may consist of an FIP primer, a BIP primer, an F3 primer and a B3 primer designed from the nucleotide sequence region as shown in SEQ ID NO: 29 in the capsulation locus region II, for example.

The FIP primer may be designed from a region ranging from bp 64 to 140 in the nucleotide sequence as shown in SEQ ID NO: 29, for example.

The BIP primer may be designed from a region ranging from bp 141 to 219 in the nucleotide sequence as shown in SEQ ID NO: 29, for example.

The F3 primer may be designed from a region ranging from bp 42 to 61 in the nucleotide sequence as shown in SEQ ID NO: 29, for example.

The B3 primer may be designed from a region ranging from bp 229 to 252 in the nucleotide sequence as shown in SEQ ID NO: 29, for example.

The LAMP primer set may be a combination of the nucleotide sequences as shown in SEQ ID NOS: 8, 9, 10 and 11, for example.

(2B) A LAMP primer set for detecting H. influenzae Type c, which comprises a combination of the nucleotide sequences as shown in SEQ ID NOS: 8, 9, 10 and 11.
(3B) A kit for detecting H. influenzae Type c, which comprises the LAMP primer set according to (2B) above.
(1C) A method of detecting H. influenzae Type d, which comprises: amplifying capsulation locus region II derived from H. influenzae Type d, using a LAMP primer set comprising one or more types of primers each having a nucleotide sequence that is identical to or complementary to a partial sequence in the nucleotide sequence region of the capsulation locus region II; and detecting the obtained amplification product.

The LAMP primer set may consist of an FIP primer, a BIP primer, an F3 primer and a B3 primer designed from the nucleotide sequence region as shown in SEQ ID NO: 30 in the capsulation locus region II, for example.

The FIP primer may be designed from a region ranging from bp 346 to 410 in the nucleotide sequence as shown in SEQ ID NO: 30, for example.

The BIP primer may be designed from a region ranging from bp 445 to 519 in the nucleotide sequence as shown in SEQ ID NO: 30, for example.

The F3 primer may be designed from a region ranging from bp 320 to 342 in the nucleotide sequence as shown in SEQ ID NO: 30, for example.

The B3 primer may be designed from a region ranging from bp 527 to 550 in the nucleotide sequence as shown in SEQ ID NO: 30, for example.

The LAMP primer set may be a combination of the nucleotide sequences as shown in SEQ ID NOS: 12, 13, 14 and 15, for example.

(2C) A LAMP primer set for detecting H. influenzae Type d, which comprises a combination of the nucleotide sequences as shown in SEQ ID NOS: 12, 13, 14 and 15.
(3C) A kit for detecting H. influenzae Type d, which comprises the LAMP primer set according to (2C) above.
(1D) A method of detecting H. influenzae Type e, which comprises: amplifying capsulation locus region II derived from H. influenzae Type e, using a LAMP primer set comprising one or more types of primers each having a nucleotide sequence that is identical to or complementary to a partial sequence in the nucleotide sequence region of the capsulation locus region II; and detecting the obtained amplification product.

The LAMP primer set may consist of an FIP primer, a BIP primer, an F3 primer and a B3 primer designed from the nucleotide sequence region as shown in SEQ ID NO: 31 in the capsulation locus region II, for example.

The FIP primer may be designed from a region ranging from bp 608 to 667 in the nucleotide sequence as shown in SEQ ID NO: 31, for example.

The BIP primer may be designed from a region ranging from bp 687 to 770 in the nucleotide sequence as shown in SEQ ID NO: 31, for example.

The F3 primer may be designed from a region ranging from bp 582 to 599 in the nucleotide sequence as shown in SEQ ID NO: 31, for example.

The B3 primer may be designed from a region ranging from bp 781 to 798 in the nucleotide sequence as shown in SEQ ID NO: 31, for example.

The LAMP primer set may be a combination of the nucleotide sequences as shown in SEQ ID NOS: 16, 17, 18 and 19, for example.

(2D) A LAMP primer set for detecting H. influenzae Type e, which comprises a combination of the nucleotide sequences as shown in SEQ ID NOS: 16, 17, 18 and 19.
(3D) A kit for detecting H. influenzae Type e, which comprises the LAMP primer set according to (2D) above.
(1E) A method of detecting H. influenzae Type f, which comprises: amplifying capsulation locus region II derived from H. influenzae Type f, using a LAMP primer set comprising one or more types of primers each having a nucleotide sequence that is identical to or complementary to a partial sequence in the nucleotide sequence region of the capsulation locus region II; and detecting the obtained amplification product.

The LAMP primer set may consist of an FIP primer, a BIP primer, an F3 primer and a B3 primer designed from the nucleotide sequence region as shown in SEQ ID NO: 33 in the capsulation locus region II, for example. Moreover, the LAMP primer set may further comprise an LF primer and/or an LB primer as a loop primer(s).

The FIP primer may be designed from a region ranging from bp 12086 to 12169 in the nucleotide sequence as shown in SEQ ID NO: 33, for example.

The BIP primer may be designed from a region ranging from bp 12184 to 12266 in the nucleotide sequence as shown in SEQ ID NO: 33, for example.

The F3 primer may be designed from a region ranging from bp 12063 to 12084 in the nucleotide sequence as shown in SEQ ID NO: 33, for example.

The B3 primer may be designed from a region ranging from bp 12281 to 12304 in the nucleotide sequence as shown in SEQ ID NO: 33, for example.

The LF primer may be designed from a region ranging from bp 12116 to 12139 in the nucleotide sequence as shown in SEQ ID NO: 33, for example.

The LB primer may be designed from a region ranging from bp 12210 to 12234, or from bp 12117 to 12139 in the nucleotide sequence as shown in SEQ ID NO: 33, for example.

The LAMP primer set may be a combination of the nucleotide sequences described in the following (a), (b) or (c), for example:

(a) a combination of the nucleotide sequences as shown in SEQ ID NOS: 20, 21, 22 and 23;
(b) a combination of the nucleotide sequences as shown in SEQ ID NOS: 20, 21, 22, 23, 24 and 25; or
(c) a combination of the nucleotide sequences as shown in SEQ ID NOS: 20, 21, 22, 23, 24 and 26.
(2E) A LAMP primer set for detecting H. influenzae Type f, which comprises a combination of the nucleotide sequences described in the following (a), (b) or (c):
(a) a combination of the nucleotide sequences as shown in SEQ ID NOS: 20, 21, 22 and 23;
(b) a combination of the nucleotide sequences as shown in SEQ ID NOS: 20, 21, 22, 23, 24 and 25; or
(c) a combination of the nucleotide sequences as shown in SEQ ID NOS: 20, 21, 22, 23, 24 and 26.
(3E) A kit for detecting H. influenzae Type f, which comprises the LAMP primer set according to (2E) above.

### BRIEF DESCRIPTION OF THE DRAWINGS

FIG. 1 is a view showing a part (SEQ ID NO: 28) of an example of the nucleotide sequence region (SEQ ID NO: 27) of capsulation gene locus region II derived from H. influenzae Type a, and an example of a target region used for designing each LAMP primer in a first embodiment of the present invention.

FIG. 2 is a view showing an example of the nucleotide sequence region (SEQ ID NO: 29) of capsulation gene locus region II derived from H. influenzae Type c, and an example of a target region used for designing each LAMP primer in a second embodiment of the present invention.

FIG. 3 is a view showing an example of the nucleotide sequence region (SEQ ID NO: 30) of capsulation gene locus region II derived from H. influenzae Type d, and an example of a target region used for designing each LAMP primer in a third embodiment of the present invention.

FIG. 4 is a view showing a part (SEQ ID NO: 32) of an example of the nucleotide sequence region (SEQ ID NO: 31) of capsulation gene locus region II derived from H. influenzae Type e, and an example of a target region used for designing each LAMP primer in a fourth embodiment of the present invention.

FIG. 5 is a view showing a part (SEQ ID NO: 34) of an example of the nucleotide sequence region (SEQ ID NO: 33) of capsulation gene locus region II derived from H. influenzae Type f, and an example of a target region used for designing each LAMP primer in a fifth embodiment of the present invention.

FIG. 6 is a view showing the results of a real-time turbidity measurement in the case of using a LAMP primer set HiA1.

FIG. 7 is a graph showing the relationship between a threshold time (Tt) in the case of using the LAMP primer set HiA1 and the common logarithm of an initial template DNA concentration.

FIG. 8 is a view showing the results of a real-time turbidity measurement in the case of using a LAMP primer set HiC1.

FIG. 9 is a graph showing the relationship between a threshold time (Tt) in the case of using the LAMP primer set HiC1 and the common logarithm of an initial template DNA concentration.

FIG. 10 is a view showing the results of a real-time turbidity measurement in the case of using a LAMP primer set HiD1.

FIG. 11 is a graph showing the relationship between a threshold time (Tt) in the case of using the LAMP primer set HiD1 and the common logarithm of an initial template DNA concentration.

FIG. 12 is a view showing the results of a real-time turbidity measurement in the case of using a LAMP primer set HiE1.

FIG. 13 is a graph showing the relationship between a threshold time (Tt) in the case of using the LAMP primer set HiE1 and the common logarithm of an initial template DNA concentration.

FIG. 14 is a view showing the results of a real-time turbidity measurement in the case of using a LAMP primer set HiF1.

FIG. 15 is a graph showing the relationship between a threshold time (Tt) in the case of using the LAMP primer set HiF1 and the common logarithm of an initial template DNA concentration.

### BEST MODE FOR CARRYING OUT THE INVENTION

The present invention will be described in detail below. The following descriptions are not intended to limit the scope of the present invention. Other than the following examples, the present invention may be modified and may be carried out, as appropriate, within a range that does not impair the intention of the present invention.

The present specification includes all of the contents as disclosed in the specification of Japanese Patent Application No. 2006-116104, which is a priority document of the present application. Moreover, all publications cited in the present specification, which include prior art documents and patent documents such as laid-open application publications or patent publications, are incorporated herein by reference in their entirety.

### 1. Summary of the Present Invention

The present invention has focused on a nucleotide sequence region specific for each capsular serotype in chromosomal DNA derived from each of capsular serotype H. influenzae other than H. influenzae Type b (namely, capsular serotypes a, c, d, e and f), so as to design a LAMP primer set, and thus the inventors have enabled specific detection (typing) of the capsular serotype H. influenzae other than the H. influenzae Type b. The term “LAMP primer set” is used herein to mean a primer set consisting of at least 4 types of (at maximum 6 types) of primers used in nucleic acid amplification according to a LAMP method (the term “LAMP primer set” has the same definition throughout the present specification).

The LAMP primer set is constituted by combining primers designed from 6 different regions (F3, F2, F1, B1c, B2c and B3c from the 5′ end side) in a nucleotide sequence region specific for each capsular serotype and 6 regions complementary thereto (B3, B2, B1, F1c, F2c and F3c from the 5′ end side). Specifically, the LAMP primer set is produced by combining: a Forward Inner Primer (hereinafter abbreviated as “FIP” at times) formed by ligating nucleotides in the F1c region to nucleotides in the F2 region from the 5′ end side of a nucleotide sequence region specific for each capsular serotype; a Backward Inner Primer (hereinafter abbreviated as “BIP” at times) formed by ligating nucleotides in the B1c region to nucleotides in the B2 region from the 5′ end side thereof; an F3 primer consisting of nucleotides in the F3 region; and a B3 primer consisting of nucleotides in the B3 region. If desired, loop primers may be further designed, and DNA may be amplified using such primers, so that an amplified product may be detected. If such loop primers are used, the time required until detection can be further reduced. Thus, the use of loop primers enables more efficient detection. As such loop primers, there can be used a Loop Primer Forward (hereinafter abbreviated as “LF” at times) consisting of nucleotides in a region between the F1c region and the F2c region and a Loop Primer Backward (hereinafter abbreviated as “LB” at times) consisting of nucleotides in a region between the B2 region and the B1 region.

In the LAMP method, an amplification reaction can be promoted only by incubation at a constant temperature capable of maintaining enzyme activity. Thus, the LAMP method does not need equipment for controlling each temperature, which is necessary for the PCR method. Accordingly, the LAMP method enables simple detection at low cost, as well as rapid detection without time loss caused by temperature change.

### 2. Detection of Various H. influenzae Types

(1) Detection of H. influenzae Type a

In a first embodiment of the present invention, a LAMP primer set is designed by focusing on the nucleotide sequence region of capsulation locus region II derived from H. influenzae Type a, so as to specifically detect the H. influenzae Type a.

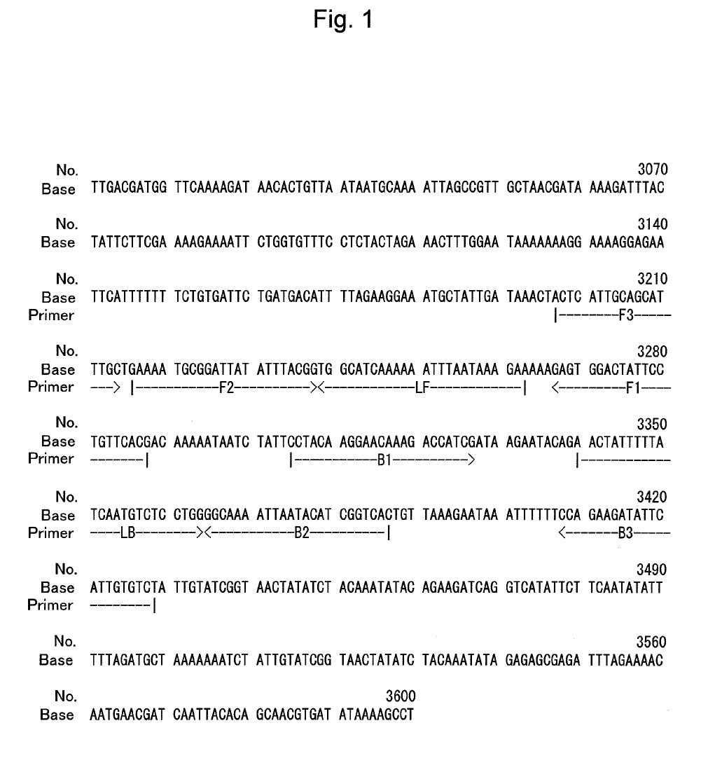

An example of the nucleotide sequence region of capsulation locus region II derived from the H. influenzae Type a is shown in FIG. 1 and SEQ ID NO: 27. Herein, the nucleotide sequence region may be either a region corresponding to the entire capsulation locus region II or a region corresponding to a portion thereof. Thus, it is not limited. It is to be noted that FIG. 1 shows only the nucleotide sequence (SEQ ID NO: 28) of a portion (bp 3001 to 3600) in the nucleotide sequence as shown in SEQ ID NO: 27. The nucleotide sequence as shown in SEQ ID NO: 27 has been published under Accession number Z37516 at GenBank (http://www.ncbi.nlm.nih.gov/).

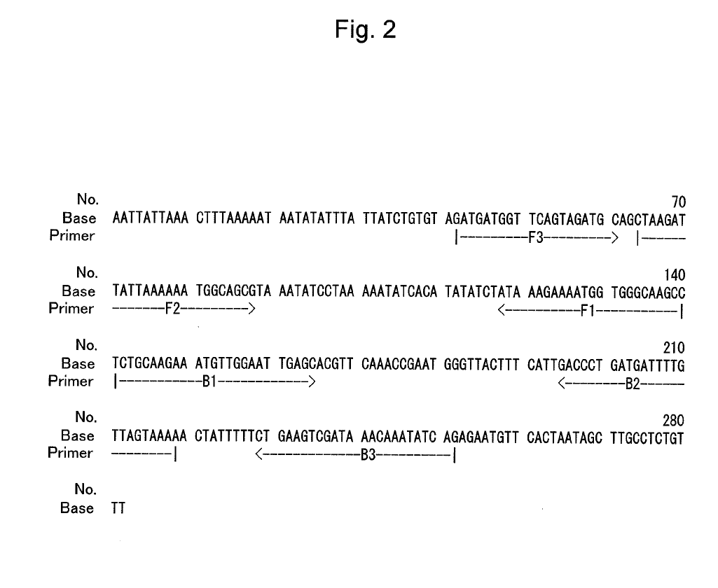

In FIG. 1, the line with the term “Number” indicates the position of nucleotides. Specifically, the number described in each line indicates the position of a nucleotide located rightmost of the nucleotide sequence immediately below the number. The line with the term “Primer” indicates an example of the position of a target region for designing the FIP, BIP, F3, B3, LF and LB primers. In addition, the line with the term “Base” indicates a nucleotide sequence in the 5′→3′ direction from the left side to the right side, as with the display method used in the sequence listing. The nucleotide located rightmost in each line leads to the nucleotide located leftmost in the nucleotide sequence in the next line. The arrow in the “Primer” line of FIG. 1 indicates the nucleotide sequence of a primer in the 5′→3′ direction. Accordingly, when a certain region is designated with a left-pointing arrow, it means that a sequence complementary to the nucleotide sequence of the region is included in the nucleotide sequence of the primer. When a certain region is designated with a right-pointing arrow, it means that the nucleotide sequence of the region is included in the nucleotide sequence of the primer. These definitions regarding FIG. 1 are also used in FIGS. 2 to 5, which will be exemplified with regard to second to fifth embodiments of the present invention.

In the first embodiment of the present invention, at least one type of (preferably at least two types of, more preferably at least four types of, and further preferably six types of) LAMP primer comprised in the LAMP primer set consists of a nucleotide sequence identical to or complementary to the sequence of a portion (partial sequence) in the nucleotide sequence of the aforementioned capsulation locus region II. Further, in the first embodiment of the present invention, the LAMP primer set comprises each LAMP primer designed preferably from the nucleotide sequence region as shown in SEQ ID NO: 27 in the aforementioned capsulation locus region II, more preferably from the nucleotide sequence region ranging from bp 3001 to 3600 in the nucleotide sequence as shown in SEQ ID NO: 27, particularly preferably from the nucleotide sequence region ranging from bp 3100 to 3500 in the nucleotide sequence as shown in SEQ ID NO: 27, and most preferably from the nucleotide sequence region ranging from bp 3197 to 3429 in the nucleotide sequence as shown in SEQ ID NO: 27. In the case of using such a LAMP primer set, the LAMP primer set is excellent in terms of detection sensitivity and detection promptness, as well as specificity, in detection of the H. influenzae Type a. Moreover, in the case of the LAMP primer set, linearity is observed in an amplification curve, and good quantitative performance can also be obtained.

In the first embodiment of the present invention, the FIP primer can be designed from the region ranging from bp 3216 to 3288 (which is hereinafter referred to as “3216-3288” at time, and the same holds for other primers) in the nucleotide sequence as shown in SEQ ID NO: 27, for example. As detailed items, F2 is preferably designed from the region of 3216-3238 (F2c is a complementary region thereof), and F1 is preferably designed from the region of 3267-3288 (SEQ ID NO: 1) (F1c is a complementary region thereof), for example.

The BIP primer can be designed from the region of 3305-3387 in the nucleotide sequence as shown in SEQ ID NO: 27, for example. As detailed items, B1c is preferably designed from the region of 3305-3327 (a complementary strand of 3305-3327 of B1), and B2 is preferably designed from the region of 3365-3387 (SEQ ID NO: 2), for example.

The F3 primer is preferably designed from the region of 3197-3214 (SEQ ID NO: 3) in the nucleotide sequence as shown in SEQ ID NO: 27, and the B3 primer is preferably designed from the region of 3408-3429 (SEQ ID NO: 4) in the nucleotide sequence as shown in SEQ ID NO: 27, for example.

In the present invention, loop primers can further be used.

The LF primer is preferably designed from the region of 3239-3263 (SEQ ID NO: 5) in the nucleotide sequence as shown in SEQ ID NO: 27, and the LB primer is preferably designed from the region of 3340-3364 (SEQ ID NO: 6) in the nucleotide sequence as shown in SEQ ID NO: 27, for example. Alternatively, the LB primer may also be designed from the region of 3339-3362 (SEQ ID NO: 7) in the nucleotide sequence as shown in SEQ ID NO: 27.

Herein, with regard to a LAMP primer set composed of the primers consisting of the aforementioned nucleotide sequences as shown in SEQ ID NOS: 1 to 6 (hereinafter referred to as HiA1 at times), the nucleotide sequences of the primers are shown in Table 1. The LB primer consisting of the nucleotide sequence as shown in SEQ ID NO: 7, which can be used as with the LB primer consisting of the nucleotide sequence as shown in SEQ ID NO: 6, is also shown in Table 1. Further, the positions of 8 target regions (F1, F2, B1, B2, F3, B3, LF, and LB) selected to design the aforementioned primers are shown in Tables 2A and 2B.

TABLE 1
LAMP primer set HiA1

| Type of | | |
|---|---|---|
| LAMP | | SEQ ID |
| primer | Nucleotide sequence | NO. |
| FIP | CGTGAACAGGAATAGTCCACTCGAAAATGCGGATT | 1 |
| | ATATTTACGG | |
| BIP | CCTACAAGGAACAAAGACCATCGGTGACCGATGTA | 2 |
| | TTAATTTTGCC | |
| F3 | ACTCATTGCAGCATTTGC | 3 |
| B3 | AGACACAATGAATATCTTCTGG | 4 |
| LF | TTCTTTATTAAATTTTTTGATGCCA | 5 |
| LB | AACTATTTTTATCAATGTCTCCTGG | 6 |
| | GAACTATTTTTATCAATGTCTCCT | 7 |

TABLE 2A

| | LAMP primer | | | | | |
|---|---|---|---|---|---|---|
| | FIP primer | | BIP primer | | F3 primer | B3 primer |
| | SEQ ID | | SEQ ID | | SEQ ID | SEQ ID |
| | NO: 1 | | NO: 2 | | NO: 3 | NO: 4 |
| bp of | F2 | F1 | B1 | B2 | F3 | B3 |
| target | 3216-3238 | 3267-3288 | 3305-3327 | 3365-3387 | 3197-3214 | 3408-3429 |
| region | | | | | | |

TABLE 2B

| LAMP primer | |
|---|---|
| LF primer | LB primer |

| | SEQ ID | SEQ ID | SEQ ID |
|---|---|---|---|
| | NO: 5 | NO: 6 | NO: 7 |
| bp of | LF | LB | LB |
| target | 3239-3263 | 3340-3364 | 3339-3362 |
| region | | | |

(2) Detection of H. influenzae Type c

In a second embodiment of the present invention, a LAMP primer set is designed by focusing on the nucleotide sequence region of capsulation locus region II derived from H. influenzae Type c, so as to specifically detect the H. influenzae Type c.

An example of the nucleotide sequence region of capsulation locus region II derived from the H. influenzae Type c is shown in FIG. 2 and SEQ ID NO: 29. Herein, the nucleotide sequence region may be either a region corresponding to the entire capsulation locus region II or a region corresponding to a portion thereof. Thus, it is not limited. The nucleotide sequence region as shown in SEQ ID NO: 29 is based on a nucleotide sequence formed by obtaining an amplification product by PCR using chromosomal DNA derived from the H. influenzae Type c as a template and also using known PCR primers published at GenBank (Accession numbers Z33387 and Z33388) and then by sequencing the obtained amplification product.

In the second embodiment of the present invention, at least one type of (preferably at least two types of, and more preferably at least four types of) LAMP primer comprised in the LAMP primer set consists of a nucleotide sequence identical to or complementary to the sequence of a portion in the nucleotide sequence of the aforementioned capsulation locus region II. Further, in the second embodiment of the present invention, the LAMP primer set comprises each LAMP primer designed preferably from the nucleotide sequence region as shown in SEQ ID NO: 29 in the aforementioned capsulation locus region II, more preferably from the nucleotide sequence region ranging from bp 5 to 280 in the nucleotide sequence as shown in SEQ ID NO: 29, particularly preferably from the nucleotide sequence region ranging from bp 30 to 270 in the nucleotide sequence as shown in SEQ ID NO: 29, and most preferably from the nucleotide sequence region ranging from bp 42 to 252 in the nucleotide sequence as shown in SEQ ID NO: 29. In the case of using such a LAMP primer set, the LAMP primer set is excellent in terms of detection sensitivity and detection promptness, as well as specificity, in detection of the H. influenzae Type c. Moreover, in the case of the LAMP primer set, linearity is observed in an amplification curve, and good quantitative performance can also be obtained.

In the second embodiment of the present invention, the FIP primer can be designed from the region of 64-140 in the nucleotide sequence as shown in SEQ ID NO: 29, for example. As detailed items, F2 is preferably designed from the region of 64-88 (F2c is a complementary region thereof), and F1 is preferably designed from the region of 118-140 (SEQ ID NO: 8) (F1c is a complementary region thereof), for example.

The BIP primer can be designed from the region of 141-219 in the nucleotide sequence as shown in SEQ ID NO: 29, for example. As detailed items, B1c is preferably designed from the region of 141-165 (a complementary strand of 141-165 of B1), and B2 is preferably designed from the region of 195-219 (SEQ ID NO: 9), for example.

The F3 primer is preferably designed from the region of 42-61 (SEQ ID NO: 10) in the nucleotide sequence as shown in SEQ ID NO: 29, and the B3 primer is preferably designed from the region of 229-252 (SEQ ID NO: 11) in the nucleotide sequence as shown in SEQ ID NO: 29, for example.

In the present invention, an LF primer and/or an LB primer may be designed and used as loop primer(s).

Herein, with regard to a LAMP primer set composed of the primers consisting of the aforementioned nucleotide sequences as shown in SEQ ID NOS: 8 to 11 (hereinafter referred to as HiC1 at times), the nucleotide sequences of the primers are shown in Table 3. Further, the positions of 6 target regions (F1, F2, B1, B2, F3, and B3) selected to design the aforementioned primers are shown in Table 4.

TABLE 3
LAMP primer set HiC1

| Type of | | |
|---|---|---|
| LAMP | | SEQ ID |
| primer | Nucleotide sequence | NO. |
| FIP | GGCTTGCCCACCATTTTCTTTATCTAAGATTATTA | 8 |
| | AAAATGGCAGCG | |
| BIP | TCTGCAAGAAATGTTGGAATTGAGCTTTTACTAAC | 9 |
| | AAAATCATCAGGGTC | |
| F3 | GATGATGGTTCAGTAGATGC | 10 |
| B3 | CTGATATTTGTTTATCGACTTCAG | 11 |

TABLE 4

| | LAMP primer | | | | | |
|---|---|---|---|---|---|---|
| | FIP primer | | BIP primer | | F3 primer | B3 primer |
| | SEQ ID | | SEQ ID | | SEQ ID | SEQ ID |
| | NO: 8 | | NO: 9 | | NO: 10 | NO: 11 |
| bp of | F2 | F1 | B1 | B2 | F3 | B3 |
| target | 64-88 | 118-140 | 141-165 | 195-219 | 42-61 | 229-252 |
| region | | | | | | |

(3) Detection of H. influenzae Type d In a third embodiment of the present invention, a LAMP primer set is designed by focusing on the nucleotide sequence region of capsulation locus region II derived from H. influenzae Type d, so as to specifically detect the H. influenzae Type d.

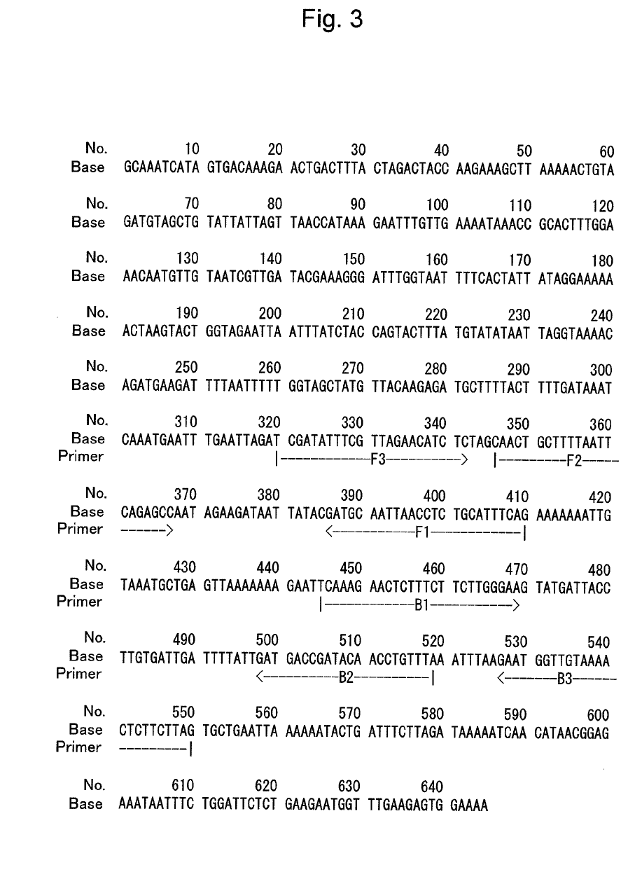

An example of the nucleotide sequence region of capsulation locus region II derived from the H. influenzae Type d is shown in FIG. 3 and SEQ ID NO: 30. Herein, the nucleotide sequence region may be either a region corresponding to the entire capsulation locus region II or a region corresponding to a portion thereof. Thus, it is not limited. The nucleotide sequence region ranging from bp 491 to 645 in the nucleotide sequence region as shown in SEQ ID NO: 30 has been published under Accession number Z33389 at GenBank.

In the third embodiment of the present invention, at least one type of (preferably at least two types of, and more preferably at least four types of) LAMP primer comprised in the LAMP primer set consists of a nucleotide sequence identical to or complementary to the sequence of a portion in the nucleotide sequence of the aforementioned capsulation locus region II. Further, in the third embodiment of the present invention, the LAMP primer set comprises each LAMP primer designed preferably from the nucleotide sequence region as shown in SEQ ID NO: 30 in the aforementioned capsulation locus region II, more preferably from the nucleotide sequence region ranging from bp 1 to 640 in the nucleotide sequence as shown in SEQ ID NO: 30, particularly preferably from the nucleotide sequence region ranging from bp 160 to 600 in the nucleotide sequence as shown in SEQ ID NO: 30, and most preferably from the nucleotide sequence region ranging from bp 320 to 550 in the nucleotide sequence as shown in SEQ ID NO: 30. In the case of using such a LAMP primer set, the LAMP primer set is excellent in terms of detection sensitivity and detection promptness, as well as specificity, in detection of the H. influenzae Type d. Moreover, in the case of the LAMP primer set, linearity is observed in an amplification curve, and good quantitative performance can also be obtained.

In the third embodiment of the present invention, the FIP primer can be designed from the region of 346-410 in the nucleotide sequence as shown in SEQ ID NO: 30, for example. As detailed items, F2 is preferably designed from the region of 346-367 (F2c is a complementary region thereof), and F1 is preferably designed from the region of 386-410 (SEQ ID NO: 12) (F1c is a complementary region thereof), for example.

The BIP primer can be designed from the region of 445-519 in the nucleotide sequence as shown in SEQ ID NO: 30, for example. As detailed items, B1c is preferably designed from the region of 445-469 (a complementary strand of 445-469 of B1), and B2 is preferably designed from the region of 498-519 (SEQ ID NO: 13), for example.

The F3 primer is preferably designed from the region of 320-342 (SEQ ID NO: 14) in the nucleotide sequence as shown in SEQ ID NO: 30, and the B3 primer is preferably designed from the region of 527-550 (SEQ ID NO: 15) in the nucleotide sequence as shown in SEQ ID NO: 30, for example.

In the present invention, an LF primer and/or an LB primer may be designed and used as loop primer(s).

Herein, with regard to a LAMP primer set composed of the primers consisting of the aforementioned nucleotide sequences as shown in SEQ ID NOS: 12 to 15 (hereinafter referred to as HiD1 at times), the nucleotide sequences of the primers are shown in Table 5. Further, the positions of 6 target regions (F1, F2, B1, B2, F3, and B3) selected to design the aforementioned primers are shown in Table 6.

TABLE 5
LAMP primer set HiD1

| Type of | | |
|---|---|---|
| LAMP | | SEQ ID |
| primer | Nucleotide sequence | NO. |
| FIP | CTGAAATGCAGAGGTTAATTGCATCCAACTGCTTT | 12 |
| | TAATTCAGAGCC | |
| BIP | TCAAAGAACTCTTTCTTCTTGGGAATAAACAGGTT | 13 |
| | GTATCGGTCATC | |
| F3 | TCGATATTTCGTTAGAACATCTC | 14 |
| B3 | CTAAGAAGAGTTTTACAACCATTC | 15 |

TABLE 6

| | LAMP primer | | | | | |
|---|---|---|---|---|---|---|
| | FIP primer | | BIP primer | | F3 primer | B3 primer |
| | SEQ ID | | SEQ ID | | SEQ ID | SEQ ID |
| | NO: 12 | | NO: 13 | | NO: 14 | NO: 15 |
| bp of | F2 | F1 | B1 | B2 | F3 | B3 |
| target | 346-367 | 386-410 | 445-469 | 498-519 | 320-342 | 527-550 |
| region | | | | | | |

(4) Detection of H. influenzae Type e

In a fourth embodiment of the present invention, a LAMP primer set is designed by focusing on the nucleotide sequence region of capsulation locus region II derived from H. influenzae Type e, so as to specifically detect the H. influenzae Type e.

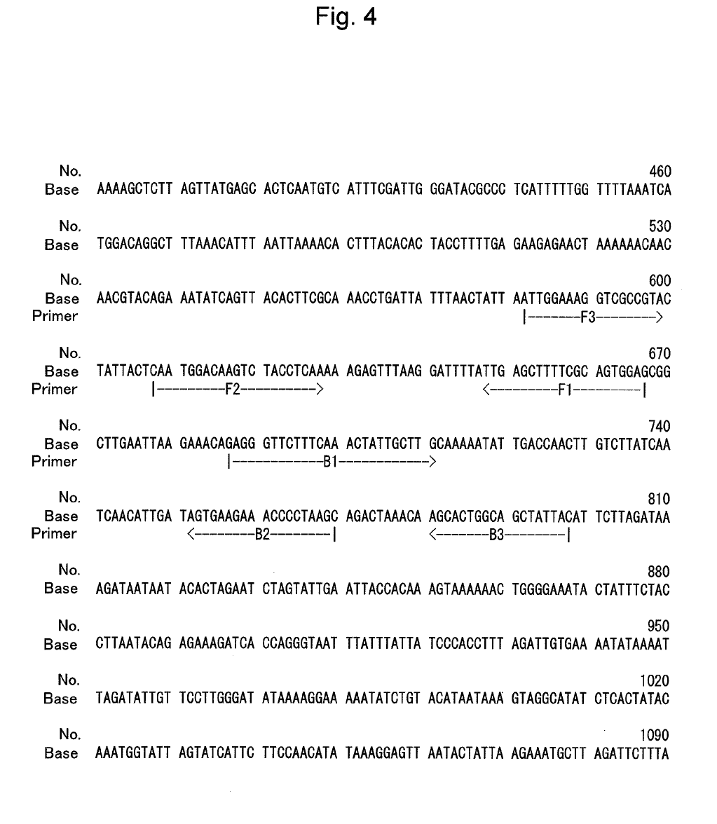

An example of the nucleotide sequence region of capsulation locus region II derived from the H. influenzae Type e is shown in FIG. 4 and SEQ ID NO: 31. Herein, the nucleotide sequence region may be either a region corresponding to the entire capsulation locus region II or a region corresponding to a portion thereof. Thus, it is not limited. It is to be noted that FIG. 4 shows only the nucleotide sequence (SEQ ID NO: 32) of a portion (bp 391 to 1090) in the nucleotide sequence as shown in SEQ ID NO: 31. The nucleotide sequence region as shown in SEQ ID NO: 31 is based on a nucleotide sequence formed by obtaining an amplification product by PCR using chromosomal DNA derived from the H. influenzae Type e as a template and also using known PCR primers published at GenBank (Accession numbers Z33390, Z33391, and Z33392) and then by sequencing the obtained amplification product.

In the fourth embodiment of the present invention, at least one type of (preferably at least two types of, and more preferably at least four types of) LAMP primer comprised in the LAMP primer set consists of a nucleotide sequence identical to or complementary to the sequence of a portion in the nucleotide sequence of the aforementioned capsulation locus region II. Further, in the fourth embodiment of the present invention, the LAMP primer set comprises each LAMP primer designed preferably from the nucleotide sequence region as shown in SEQ ID NO: 31 in the aforementioned capsulation locus region II, more preferably from the nucleotide sequence region ranging from bp 400 to 1000 in the nucleotide sequence as shown in SEQ ID NO: 31, particularly preferably from the nucleotide sequence region ranging from bp 500 to 900 in the nucleotide sequence as shown in SEQ ID NO: 31, and most preferably from the nucleotide sequence region ranging from bp 582 to 798 in the nucleotide sequence as shown in SEQ ID NO: 31. In the case of using such a LAMP primer set, the LAMP primer set is excellent in terms of detection sensitivity and detection promptness, as well as specificity, in detection of the H. influenzae Type e. Moreover, in the case of the LAMP primer set, linearity is observed in an amplification curve, and good quantitative performance can also be obtained.

In the fourth embodiment of the present invention, the FIP primer can be designed from the region of 608-667 in the nucleotide sequence as shown in SEQ ID NO: 31, for example. As detailed items, F2 is preferably designed from the region of 608-628 (F2c is a complementary region thereof), and F1 is preferably designed from the region of 648-667 (SEQ ID NO: 16) (F1c is a complementary region thereof), for example.

The BIP primer can be designed from the region of 687-770 in the nucleotide sequence as shown in SEQ ID NO: 31, for example. As detailed items, B1c is preferably designed from the region of 687-711 (a complementary strand of 687-711 of B1), and B2 is preferably designed from the region of 752-770 (SEQ ID NO: 17), for example.

The F3 primer is preferably designed from the region of 582-599 (SEQ ID NO: 18) in the nucleotide sequence as shown in SEQ ID NO: 31, and the B3 primer is preferably designed from the region of 781-798 (SEQ ID NO: 19) in the nucleotide sequence as shown in SEQ ID NO: 31, for example.

In the present invention, an LF primer and/or an LB primer may be designed and used as loop primer(s).

Herein, with regard to a LAMP primer set composed of the primers consisting of the aforementioned nucleotide sequences as shown in SEQ ID NOS: 16 to 19 (hereinafter referred to as HiE1 at times), the nucleotide sequences of the primers are shown in Table 7. Further, the positions of 6 target regions (F1, F2, B1, B2, F3, and B3) selected to design the aforementioned primers are shown in Table 8.

TABLE 7
LAMP primer set HiE1

| Type of | | |
|---|---|---|
| LAMP | | SEQ ID |
| primer | Nucleotide sequence | NO. |
| FIP | CTCCACTGCGAAAAGCTCAACAATGGACAAGTCTA | 16 |
| | CCTCAA | |
| BIP | GAGGGTTCTTTCAAACTATTGCTTGGCTTAGGGGT | 17 |
| | TTCTTCACT | |
| F3 | ATTGGAAAGGTCGCCGTA | 18 |
| B3 | GTAATAGCTGCCAGTGCT | 19 |

TABLE 8

| | LAMP primer | | | | | |
|---|---|---|---|---|---|---|
| | FIP primer | | BIP primer | | F3 primer | B3 primer |
| | SEQ ID | | SEQ ID | | SEQ ID | SEQ ID |
| | NO: 16 | | NO: 17 | | NO: 18 | NO: 19 |
| bp of | F2 | F1 | B1 | B2 | F3 | B3 |
| target | 608-628 | 648-667 | 687-711 | 752-770 | 582-599 | 781-798 |
| region | | | | | | |

(5) Detection of H. influenzae Type f

In a fifth embodiment of the present invention, a LAMP primer set is designed by focusing on the nucleotide sequence region of capsulation locus region II derived from H. influenzae Type f, so as to specifically detect the H. influenzae Type f.

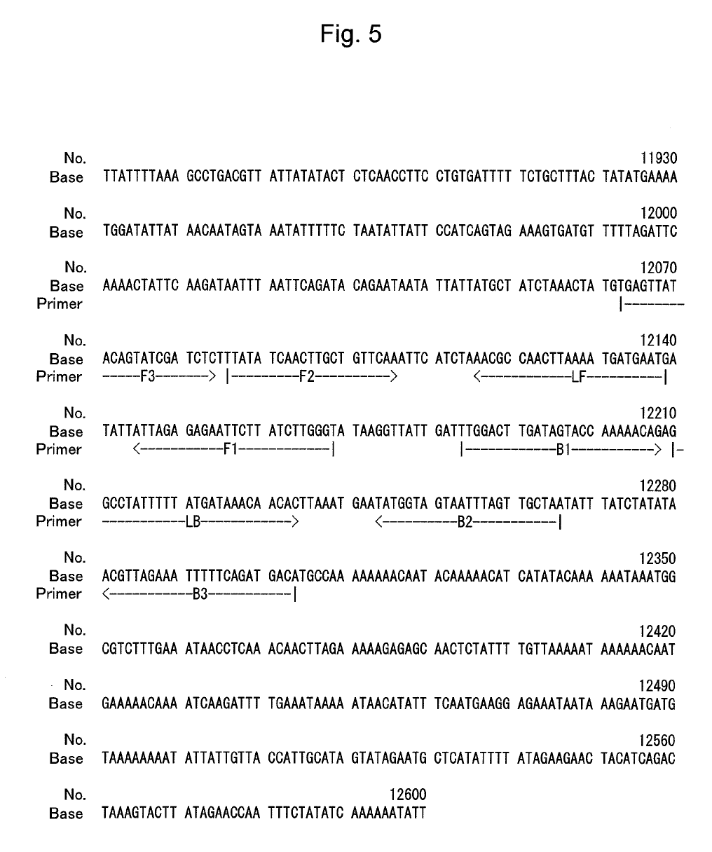

An example of the nucleotide sequence region of capsulation locus region II derived from the H. influenzae Type f is shown in FIG. 5 and SEQ ID NO: 33. Herein, the nucleotide sequence region may be either a region corresponding to the entire capsulation locus region II or a region corresponding to a portion thereof. Thus, it is not limited. It is to be noted that FIG. 5 shows only the nucleotide sequence (SEQ ID NO: 34) of a portion (bp 11861 to 12600) in the nucleotide sequence as shown in SEQ ID NO: 33. The nucleotide sequence as shown in SEQ ID NO: 33 has been published under Accession number AF549211 at GenBank.

In the fifth embodiment of the present invention, at least one type of (preferably at least two types of, more preferably at least four types of, and further preferably six types of) LAMP primer comprised in the LAMP primer set consists of a nucleotide sequence identical to or complementary to the sequence of a portion in the nucleotide sequence of the aforementioned capsulation locus region II. Further, in the fifth embodiment of the present invention, the LAMP primer set comprises each LAMP primer designed preferably from the nucleotide sequence region as shown in SEQ ID NO: 33 in the aforementioned capsulation locus region II, more preferably from the nucleotide sequence region ranging from bp 11900 to 12500 in the nucleotide sequence as shown in SEQ ID NO: 33, particularly preferably from the nucleotide sequence region ranging from bp 12000 to 12400 in the nucleotide sequence as shown in SEQ ID NO: 33, and most preferably from the nucleotide sequence region ranging from bp 12063 to 12304 in the nucleotide sequence as shown in SEQ ID NO: 33. In the case of using such a LAMP primer set, the LAMP primer set is excellent in terms of detection sensitivity and detection promptness, as well as specificity, in detection of the H. influenzae Type f. Moreover, in the case of the LAMP primer set, linearity is observed in an amplification curve, and good quantitative performance can also be obtained.

In the fifth embodiment of the present invention, the FIP primer can be designed from the region of 12086-12169 in the nucleotide sequence as shown in SEQ ID NO: 33, for example. As detailed items, F2 is preferably designed from the region of 12086-12106 (F2c is a complementary region thereof), and F1 is preferably designed from the region of 12145-12169 (SEQ ID NO: 20) (F1c is a complementary region thereof), for example.

The BIP primer can be designed from the region of 12184-12266 in the nucleotide sequence as shown in SEQ ID NO: 33, for example. As detailed items, B1c is preferably designed from the region of 12184-12208 (a complementary strand of 12184-12208 of B1), and B2 is preferably designed from the region of 12244-12266 (SEQ ID NO: 21), for example.

The F3 primer is preferably designed from the region of 12063-12084 (SEQ ID NO: 22) in the nucleotide sequence as shown in SEQ ID NO: 33, and the B3 primer is preferably designed from the region of 12281-12304 (SEQ ID NO: 23) in the nucleotide sequence as shown in SEQ ID NO: 33, for example.

In the present invention, loop primers can further be used.

The LF primer is preferably designed from the region of 12116-12139 (SEQ ID NO: 24) in the nucleotide sequence as shown in SEQ ID NO: 33, and the LB primer is preferably designed from the region of 12210-12234 (SEQ ID NO: 25) in the nucleotide sequence as shown in SEQ ID NO: 33, for example. Alternatively, the LB primer may also be designed from the region of 12117-12139 (SEQ ID NO: 26) in the nucleotide sequence as shown in SEQ ID NO: 33.

Herein, with regard to a LAMP primer set composed of the primers consisting of the aforementioned nucleotide sequences as shown in SEQ ID NOS: 20 to 25 (hereinafter referred to as HiF1 at times), the nucleotide sequences of the primers are shown in Table 9. The LB primer consisting of the nucleotide sequence as shown in SEQ ID NO: 26, which can be used as with the LB primer consisting of the nucleotide sequence as shown in SEQ ID NO: 25, is also shown in Table 9. Further, the positions of 8 target regions (F1, F2, B1, B2, F3, B3, LF, and LB) selected to design the aforementioned primers are shown in Tables 10A and 10B.

TABLE 9
LAMP primer set HiF1

| Type of | | |
|---|---|---|
| LAMP | | SEQ ID |
| primer | Nucleotide sequence | NO. |
| FIP | ACCCAAGATAAGAATTCTCTCTAATTTATATCAAC | 20 |
| | TTGCTGTTCAA | |
| BIP | TTGGACTTGATAGTACCAAAAACAGTTAGCAACTA | 21 |
| | AATTACTACCATA | |
| F3 | TGAGTTATACAGTATCGATCTC | 22 |
| B3 | TGTCATCTGAAAAATTTCTAACGT | 23 |
| LF | CATTCATCATTTTAAGTTGGCGTT | 24 |
| LB | GGCCTATTTTTATGATAAACAACAC | 25 |
| | CATTCATCATTTTAAGTTGGCGT | 26 |

TABLE 10A

| | LAMP primer | | | | | |
|---|---|---|---|---|---|---|
| | FIP primer | | BIP primer | | F3 primer | B3 primer |
| | SEQ ID | | SEQ ID | | SEQ ID | SEQ ID |
| | NO: 20 | | NO: 21 | | NO: 22 | NO: 23 |
| bp of | F2 | F1 | B1 | B2 | F3 | B3 |
| target | 12086-12106 | 12145-12169 | 12184-12208 | 12244-12266 | 12063-12084 | 12281-12304 |
| region | | | | | | |

TABLE 10B

| LAMP primer | |
|---|---|
| LF primer | LB primer |

| | SEQ ID | SEQ ID | SEQ ID |
|---|---|---|---|
| | NO: 24 | NO: 25 | NO: 26 |
| bp of | LF | LB | LB |
| target | 12116-12139 | 12210-12234 | 12117-12139 |
| region | | | |

### (6) Preparation of LAMP Primers, Detection Operations, etc.

The LAMP primers used in the first to fifth embodiments of the present invention can be prepared by chemical synthesis using a DNA automatic synthesizer, for example. In the present invention, each LAMP primer means an oligonucleotide, which has a certain nucleotide sequence as described above, is capable of forming a base pair with another nucleotide, and comprises an —OH group acting as a base point of complementary strand synthesis at the 3′ end thereof. Thus, as far as such conditions are satisfied, the backbone thereof is not necessarily limited to that due to a phosphodiester bond. For example, it may be a phosphothioate form that has not P but S as a backbone or a peptide nucleic acid based on a peptide bond.

In the first to fifth embodiments of the present invention, the type of a template-dependent nucleic acid synthase that can be used in the LAMP method is not particularly limited, as long as it has strand displacement activity. Examples of such an enzyme include Bst DNA polymerase (large fragment), Bca(exo-) DNA polymerase, E. coli DNA polymerase I Klenow fragment, Vent(Exo-) DNA polymerase (obtained by eliminating exonuclease activity from Vent DNA polymerase), DeepVent(Exo-) DNA polymerase (obtained by eliminating exonuclease activity from DeepVent DNA polymerase), and KOD DNA polymerase. Of these, Bst DNA polymerase (large fragment) is preferable. When such Bst DNA polymerase is used, the reaction is preferably carried out at a reaction optimum temperature that is between approximately 60° C. and 65° C.

Moreover, known techniques can be applied to detect an amplification product. For example, a labeled oligonucleotide that specifically recognizes an amplified gene sequence is used, or an amplification product can easily be detected also by directly subjecting a reaction solution obtained after termination of the reaction to agarose electrophoresis. Furthermore, a LAMP primer used in the present invention may also be allowed to bind to a solid phase, as with a DNA chip. When such a solid phase primer is used as a synthesis initiation point, a nucleic acid synthesis reaction product is captured by a solid phase, and thus, separation and detection can be easily carried out.

Further, since an amplification reaction is efficiency carried out at an accelerated rate by the LAMP method, amplification can be confirmed by previously adding into a reaction solution, an intercalator specifically incorporated into a molecule of double-stranded nucleic acid, such as ethidium bromide or SYBR (registered trade mark) Green I. Still further, in the LAMP method, a large amount of substrate is consumed by the synthesis of nucleic acid, and pyrophosphoric acid generated as a by-product reacts with co-existing magnesium, so that it is converted to magnesium pyrophosphate. As a result, the reaction solution becomes clouded to such an extent that it can be confirmed by naked eye. Such a clouded state is observed after completion of the reaction, or an increase in such turbidity is observed over time (in real time) from initiation of the reaction, so that amplification can be confirmed. When the clouded state is confirmed over time, a change in absorbance at 650 nm may be observed using an optical measurement apparatus (e.g. a spectrophotometer, etc.), for example. In addition, according to such a method of confirming the clouded state over time, it is also possible to quantify the amount of chromosomal DNA (the amount of template DNA) derived from each capsular serotype H. influenzae in a test sample.

### 3. Detection Kit

Various types of reagents necessary for an amplification reaction by the LAMP method have previously been packaged, and thus they can be provided in the form of a kit for detecting various H. influenzae Types. Specifically, the kit of the present invention includes the aforementioned LAMP primer set used in detection of various H. influenzae Types. In addition to such a LAMP primer set, the kit of the present invention may also comprise, as necessary, reagents necessary for detection of a synthetic reaction product, such as dNTP used as a substrate for the synthesis of a complementary strand, DNA polymerase used in the synthesis of a strand-displacement-type complementary strand, and a buffer solution that provides conditions preferable for an enzyme reaction. Moreover, the present kit may further comprise a reagent (e.g. betaine, etc.) for destabilizing the double strand of nucleic acid.

Hereafter, the present invention will be more specifically described in the following examples. However, these examples are not intended to limit the scope of the present invention.

### EXAMPLES

### Example 1

### Specificity Confirmation Test

The detection method of the present invention was carried out and the specificity was confirmed. The details will be described below.

### (1) Preparation of Chromosomal DNA

First, chromosomal DNA was purified from various types of strains that were to be subjected to a test, and DNA used as a template of an amplification reaction was prepared.

Such chromosomal DNA was obtained by extracting it from various types of strains employing Dr. GenTLE (registered trade mark; manufactured by Takara Bio Inc.) used for enzymes, and then purifying it using QIAmp (registered trade mark) DNA minikit (manufactured by Qiagen). Extraction and purification operations were carried out in accordance with manuals included with the kits.

In the present test, chromosomal DNAs were extracted from total 28 strains (7 types of H. influenzae and 21 strains other than H. influenzae), and they were used. The 28 strains are shown in the following Table 11.

TABLE 11

| | LAMP primer set | | | | |
|---|---|---|---|---|---|
| Strain name | HiA1 | HiC1 | HiD1 | HiE1 | HiF1 |
| Streptococcus mitis ATCC9811 | − | − | − | − | − |
| Streptococcus oralis ATCC10557 | − | − | − | − | − |
| Streptococcus gordonii ATCC10558 | − | − | − | − | − |
| Streptococcus mutans XC47 | − | − | − | − | − |
| Streptococcus sanguis ATCC10556 | − | − | − | − | − |
| Streptococcus salivarius HHT | − | − | − | − | − |
| Streptococcus pneumoniae ATCC6305 | − | − | − | − | − |
| Streptococcus pneumoniae R6 | − | − | − | − | − |
| Streptococcus pneumoniae GTC261 | − | − | − | − | − |
| Streptococcus pneumoniae IID553 | − | − | − | − | − |
| Streptococcus pneumoniae IID554 | − | − | − | − | − |
| Escherichia coli DH5α | − | − | − | − | − |
| Actinobacillus actinomycetemcomitans Y4 | − | − | − | − | − |
| Porphyromonas gingivalis 381 | − | − | − | − | − |
| Porphyromonas gingivalis ATCC49417 | − | − | − | − | − |
| Actinomyces naeslundii WVU627 | − | − | − | − | − |
| Prevotella intermedia ATCC25611 | − | − | − | − | − |
| Prevotella nigrescens ATCC25261 | − | − | − | − | − |
| Haemophilus parainfluenzae IID991 | − | − | − | − | − |
| Haemophilus parahaemolyticus GTC1529 | − | − | − | − | − |
| Haemophilus aegyptius IID993 | − | − | − | − | − |
| Haemophilus influenzae type a IID983 | + | − | − | − | − |
| Haemophilus influenzae type b IID984 | − | − | − | − | − |
| Haemophilus influenzae type c IID985 | − | + | − | − | − |
| Haemophilus influenzae type d IID986 | − | − | + | − | − |
| Haemophilus influenzae type e IID987 | − | − | − | + | − |
| Haemophilus influenzae type f IID988 | − | − | − | − | + |
| Haemophilus influenzaenontypeable IID989 | − | − | − | − | − |

### (2) Concerning LAMP Reaction

Next, the LAMP primer sets (HiA1, HiC1, HiD1, HiE1, and HiF1) shown in the aforementioned Tables 1, 3, 5, 7, and 9 were used, and a LAMP reaction was carried out using, as a template, the chromosomal DNA derived from various types of strains purified in (1) above.

A LAMP reaction (25 μl) was prepared by mixing an FIP primer and a BIP primer (1.6 μM each), an F3 primer and a B3 primer (0.2 μM each), an LF primer and an LB primer (0.4 μM each) (only in the case of the LAMP primer sets HiA1 and HiF1), a 8U Bst DNA polymerase large fragment (manufactured by New England Biolabs) and deoxynucleoside triphosphate (1.4 mM each), betaine (0.8 M), a Tris-HCl buffer solution (pH 8.8; 20 mM), KCl (10 mM), (NH₄)₂SO₄ (10 mM), MgSO₄ (8 mM), 0.1% Tween 20, and 2 μl of the template DNA solution purified in (1) above (template DNA concentration: approximately 10⁶ copies).

Thereafter, this LAMP reaction solution was incubated at 63° C. for 60 minutes to promote the LAMP reaction, and finally, the solution was heated at 80° C. for 2 minutes so as to terminate the reaction.

### (3) Concerning Confirmation of Presence or Absence of Amplification

The presence or absence of amplification was detected by directly looking at the reaction tube by naked eye and observing the presence or absence of cloudiness of the LAMP reaction solution. That is to say, magnesium pyrophosphate is generated as a by-product of the reaction in an amount proportional to the amount of a replication sequence when such a replication sequence is present, and as a result, the LAMP reaction solution becomes clouded. On the other hand, when such a replication sequence is not present, the LAMP reaction solution remains transparent. Thus, an amplification product was detected using such cloudiness as an indicator.

Moreover, the presence or absence of amplification was also confirmed by subjecting the amplification product to agarose gel electrophoresis (3% agarose gel; ethidium bromide staining). As a result, a replication sequence appeared as a ladder-like pattern characteristic for the LAMP reaction (not shown in figures).

### (4) Concerning Test Results

The results of the aforementioned test are shown in Table 11 (as shown above). With regard to the results, a case where amplification (cloudiness) was confirmed by visual observation after incubation for 60 minutes was expressed with the symbol “+,” and a case where such amplification was not confirmed after such incubation for 60 minutes was expressed with the symbol “−”

As a result, in the case of using the LAMP primer set HiA1, a large amount of amplification product was confirmed only when chromosomal DNA derived from H. influenzae Type a was used as a template. In contrast, in the case of using other types of strains, no amplification products were confirmed.

In the case of using the LAMP primer set HiC1, a large amount of amplification product was confirmed only when chromosomal DNA derived from H. influenzae Type c was used as a template. In contrast, in the case of using other types of strains, no amplification products were confirmed.

In the case of using the LAMP primer set HiD1, a large amount of amplification product was confirmed only when chromosomal DNA derived from H. influenzae Type d was used as a template. In contrast, in the case of using other types of strains, no amplification products were confirmed.

In the case of using the LAMP primer set HiE1, a large amount of amplification product was confirmed only when chromosomal DNA derived from H. influenzae Type e was used as a template. In contrast, in the case of using other types of strains, no amplification products were confirmed.

In the case of using the LAMP primer set HiF1, a large amount of amplification product was confirmed only when chromosomal DNA derived from H. influenzae Type f was used as a template. In contrast, in the case of using other types of strains, no amplification products were confirmed.

From these results, it was confirmed that the method of detecting various H. influenzae Types of the present invention is excellent in terms of specificity and that it is useful as a typing method.

### Example 2

### Sensitivity Confirmation Test

Detection sensitivity obtained using various LAMP primer sets (HiA1, HiC1, HiD1, HiE1 and HiF1) was confirmed. The details will be described below.

### (1) Preparation of Chromosomal DNA

In the present test, chromosomal DNA was purified from various H. influenzae Types (capsular serotype a (IID983), capsular serotype c (IID985), capsular serotype d (IID986), capsular serotype e (IID987), and capsular serotype f (IID988)) by the same method as that described in Example 1 (1) above. The purified chromosomal DNA was used as a template. The concentration of template DNA in the reaction solution (copy number) was quantified at a molecular size of 1.9 Mbp using Ultrospec 3300 pro (manufactured by Amersham Biosciences).

### (2) Concerning LAMP Method and PCR Method

The template DNA solution quantified in (1) above was repeatedly diluted by a factor of 10, so as to prepare solutions diluted by a factor of 1 to 1,000,000. The thus prepared solutions were used as template DNA solutions in the LAMP reaction, so that a detection limit was confirmed. On the other hand, as a negative control, the detection limit of a solution with a template DNA concentration of 0 was also examined. In terms of the additive amount of a template DNA solution and the additive amounts of other additives, the same LAMP reaction solution as that used in the specificity confirmation test of Example 1 was used, with the exception that the concentration of the template DNA solution was different. Moreover, the LAMP reaction was promoted by incubating the reaction solution at 63° C. for 35 minutes or 60 minutes, and finally, the solution was heated at 80° C. for 2 minutes so as to terminate the reaction.

### (3) Concerning Confirmation of Presence or Absence of Amplification

With regard to the presence or absence of amplification by the LAMP reaction, turbidity was measured over time using a Loopamp (registered trade mark) real-time turbidity measurement apparatus (manufactured by Teramecs Co., Ltd.; model No. LA-200), and when the turbidity became 0.1 or greater, it was determined that an amplification product was confirmed.

Furthermore, as in the case of the specificity confirmation test of Example 1, the presence or absence of amplification was also confirmed by visual observation and 3% agarose gel electrophoresis (not shown in figures).

### (4) Concerning Test Results

With regard to test results, as described above, a case where an amplification product was confirmed was expressed with the symbol “+,” and a case where such an amplification product was not confirmed was expressed with the symbol “−.” The test results are shown in the following Table 12.

TABLE 12

| | Template DNA concentration (copy number) | | | | | | | |
|---|---|---|---|---|---|---|---|---|
| LAMPprimer set | 10⁶ | 10⁵ | 10⁴ | 10³ | 10² | 10 | 1 | 0 |
| HiA1 (35 min) | + | + | + | + | + | − | − | − |
| HiA1 (60 min) | + | + | + | + | + | − | − | − |
| HiC1 (35 min) | + | − | − | − | − | − | − | − |
| HiC1 (60 min) | + | + | + | + | − | − | − | − |
| HiD1 (35 min) | + | + | + | + | − | − | − | − |
| HiD1 (60 min) | + | + | + | + | + | + | − | − |
| HiE1 (35 min) | + | − | − | − | − | − | − | − |
| HiE1 (60 min) | + | + | + | + | + | − | − | − |
| HiF1 (35 min) | + | + | + | + | + | − | − | − |
| HiF1 (60 min) | + | + | + | + | + | + | − | − |

As shown in Table 12, in all cases of using LAMP primer sets HiA1, HiC1, HiD1, HiE1, and HiF1, 10³ copies of template DNA could be detected by performing the LAMP reaction for 60 minutes. Among others, HiE1 and HiA1 were able to detect 102 copies of template DNA, and HiD1 and HiF1 were able to detect 10 copies of template DNA. Thus, their sensitivity was particularly high.

Further, even if the LAMP reaction time was 35 minutes, HiA1 and HiF1 were able to detect 10² copies of template DNA, and thus it was confirmed that they were excellent in terms of both sensitivity and promptness. Since HiA1 and HiF1 also comprise loop primers, it is considered that these primer sets brought on the aforementioned results that were superior to those of HiC1 and HiE1.

### Example 3

### Real-Time Turbidity Measurement Test

With regard to the LAMP reaction using each LAMP primer set (HiA1, HiC1, HiD1, HiE1, and HiF1), a real-time turbidity measurement was carried out, and the quantitative performance of template DNA was analyzed.

In the present test, the template DNA concentration per reaction tube was adjusted to 0 to 10⁶ copies, and the LAMP reaction was then carried out using each of the aforementioned primer sets. During the reaction, absorbance at 650 nm was measured every 6 seconds using a Loopamp (registered trade mark) real-time turbidity measurement apparatus (manufactured by Teramecs Co., Ltd.; model No. LA-200).

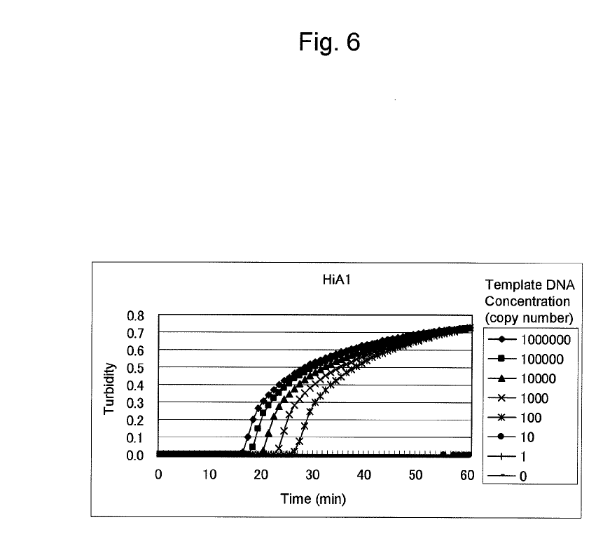

The results of a real-time turbidity measurement in the case of using the LAMP primer set HiA1 are shown in FIG. 6. As shown in FIG. 6, it was confirmed that the turbidity became 0.1 or greater within 60 minutes if the template DNA concentration was 10² copies or more. This result corresponded to the result of the confirmation of the presence or absence of amplification by visual observation and electrophoresis in the sensitivity test of Example 2. Furthermore, it was confirmed that the threshold time (the time required until the turbidity exceeded 0.1) became shorter, as the concentration of the initially used template DNA was increased.

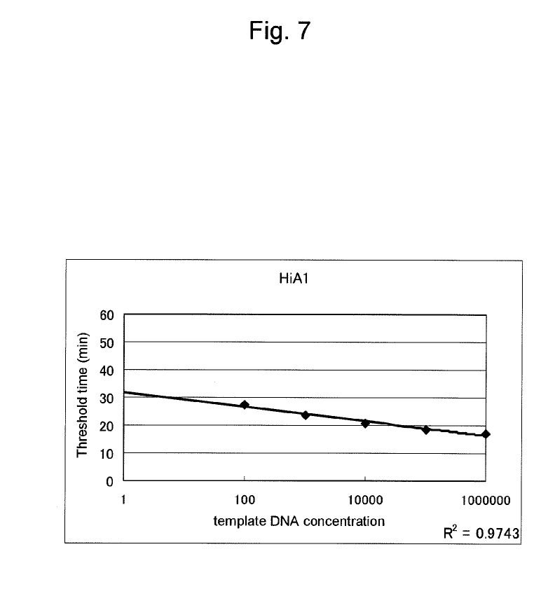

The graph as shown in FIG. 7 shows the relationship between the threshold time (Tt) obtained in the case of using HiA1 and the common logarithm of the initial template DNA concentration. A linearity was observed between these factors, and a high correlation (correlation coefficient r²=0.9743) was shown. This means that not only the presence of template DNA but also the concentration thereof can be quantified when the initial concentration of the template DNA is unknown. That is to say, even in the case of a sample whose concentration is unknown, for example, if diluted solutions of different dilution magnifications are prepared, a LAMP reaction is then carried out using each diluted solution, and a threshold time is then measured in each LAMP reaction so as to produce a regression line based on such threshold times, the unknown initial concentration of the template DNA can be determined.

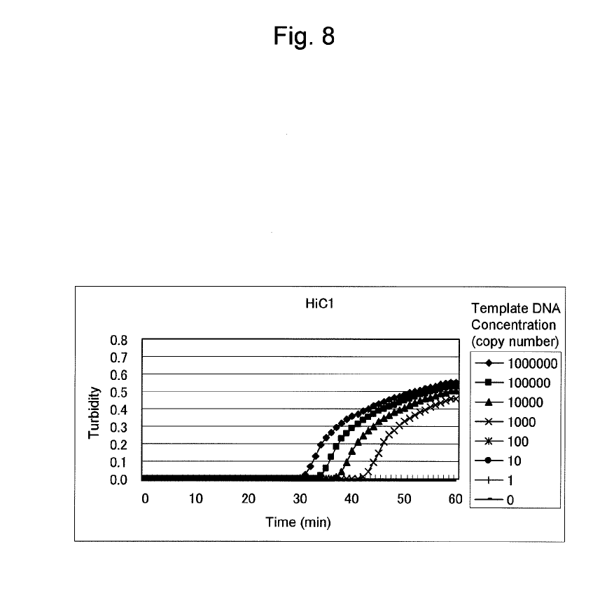

The results of a real-time turbidity measurement in the case of using the LAMP primer set HiC1 are shown in FIG. 8. As shown in FIG. 8, it was confirmed that the turbidity became 0.1 or greater within 60 minutes if the template DNA concentration was 10³ copies or more. This result corresponded to the result of the confirmation of the presence or absence of amplification by visual observation and electrophoresis in the sensitivity test of Example 2. Furthermore, it was confirmed that the threshold time (the time required until the turbidity exceeded 0.1) became shorter, as the concentration of the initially used template DNA was increased.

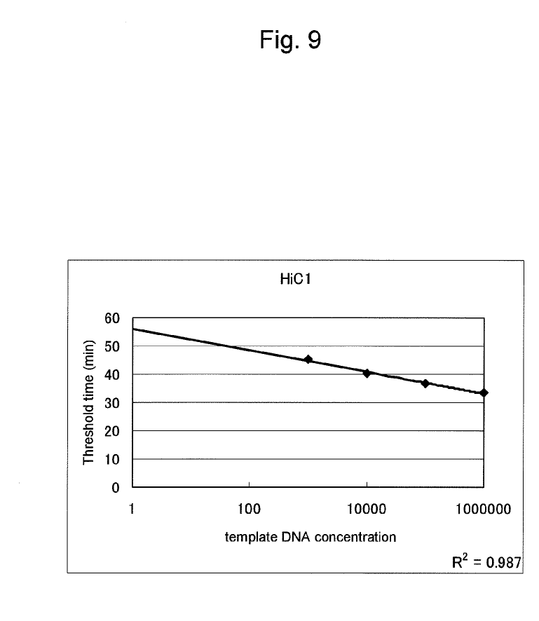

The graph as shown in FIG. 9 shows the relationship between the threshold time (Tt) obtained in the case of using HiC1 and the common logarithm of the initial template DNA concentration. A linearity was observed between these factors, and a high correlation (correlation coefficient r²=0.987) was shown. This means that not only the presence of template DNA but also the concentration thereof can be quantified when the initial concentration of the template DNA is unknown.

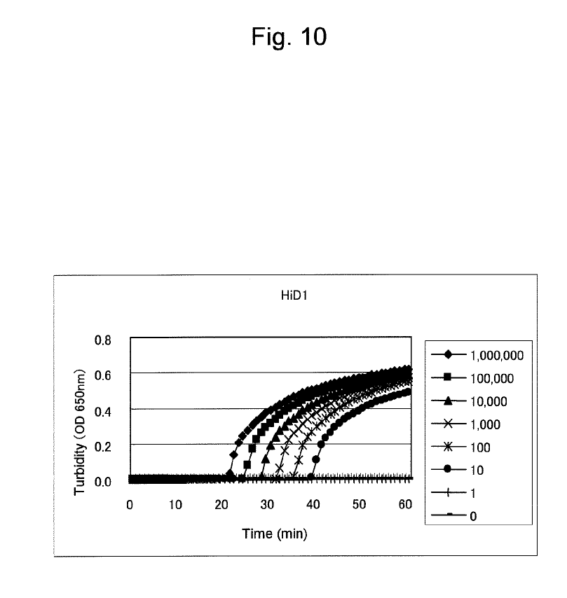

The results of a real-time turbidity measurement in the case of using the LAMP primer set HiD1 are shown in FIG. 10. As shown in FIG. 10, it was confirmed that the turbidity became 0.1 or greater within 60 minutes if the template DNA concentration was 10 copies or more. This result corresponded to the result of the confirmation of the presence or absence of amplification by visual observation and electrophoresis in the sensitivity test of Example 2. Furthermore, it was confirmed that the threshold time (the time required until the turbidity exceeded 0.1) became shorter, as the concentration of the initially used template DNA was increased.

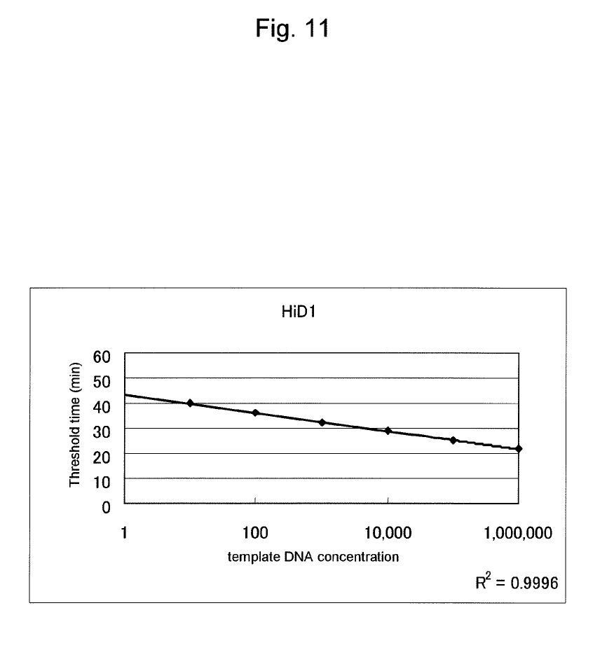

The graph as shown in FIG. 11 shows the relationship between the threshold time (Tt) obtained in the case of using HiD1 and the common logarithm of the initial template DNA concentration. A linearity was observed between these factors, and a high correlation (correlation coefficient r²=0.999) was shown. This means that not only the presence of template DNA but also the concentration thereof can be quantified when the initial concentration of the template DNA is unknown.

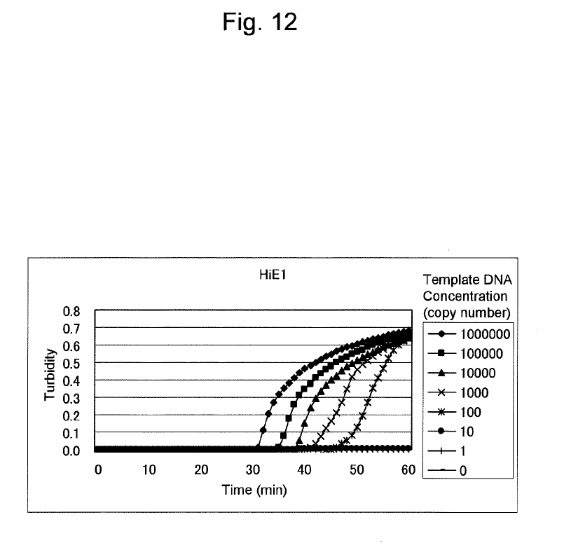

The results of a real-time turbidity measurement in the case of using the LAMP primer set HiE1 are shown in FIG. 12. As shown in FIG. 12, it was confirmed that the turbidity became 0.1 or greater within 60 minutes if the template DNA concentration was 10² copies or more. This result corresponded to the result of the confirmation of the presence or absence of amplification by visual observation and electrophoresis in the sensitivity test of Example 2. Furthermore, it was confirmed that the threshold time (the time required until the turbidity exceeded 0.1) became shorter, as the concentration of the initially used template DNA was increased.

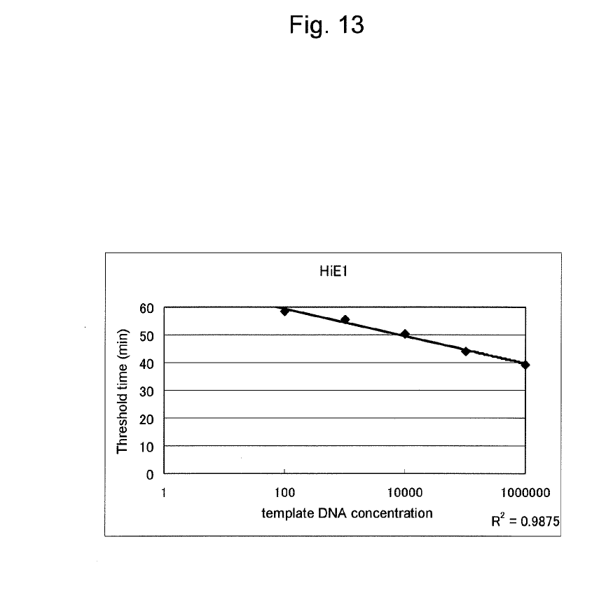

The graph as shown in FIG. 13 shows the relationship between the threshold time (Tt) obtained in the case of using HiE1 and the common logarithm of the initial template DNA concentration. A linearity was observed between these factors, and a high correlation (correlation coefficient r²=0.9875) was shown. This means that not only the presence of template DNA but also the concentration thereof can be quantified when the initial concentration of the template DNA is unknown.

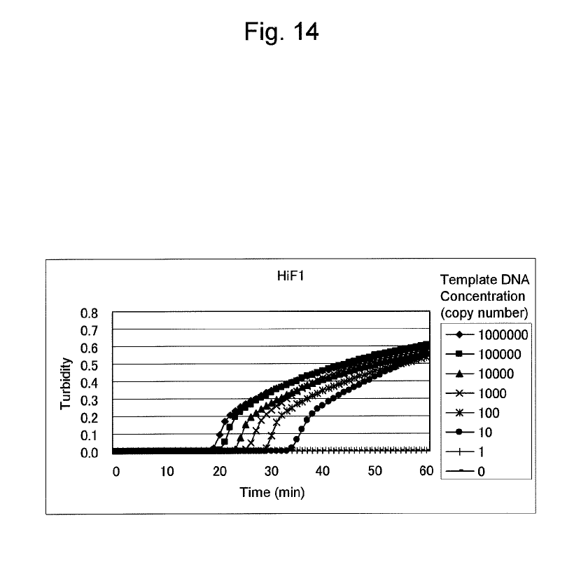

The results of a real-time turbidity measurement in the case of using the LAMP primer set HiF1 are shown in FIG. 14. As shown in FIG. 14, it was confirmed that the turbidity became 0.1 or greater within 60 minutes if the template DNA concentration was 10 copies or more. This result corresponded to the result of the confirmation of the presence or absence of amplification by visual observation and electrophoresis in the sensitivity test of Example 2. Furthermore, it was confirmed that the threshold time (the time required until the turbidity exceeded 0.1) became shorter, as the concentration of the initially used template DNA was increased.

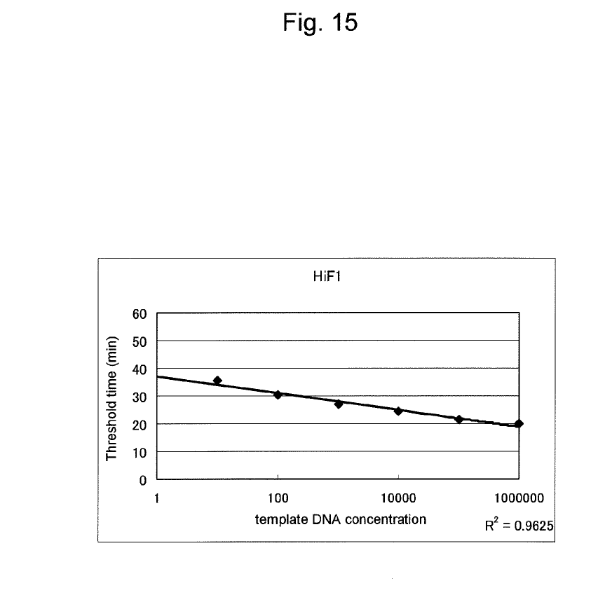

The graph as shown in FIG. 15 shows the relationship between the threshold time (Tt) obtained in the case of using HiF1 and the common logarithm of the initial template DNA concentration. A linearity was observed between these factors, and a high correlation (correlation coefficient r²=0.9625) was shown. This means that not only the presence of template DNA but also the concentration thereof can be quantified when the initial concentration of the template DNA is unknown.

From the aforementioned results, it was found that the use of any of the LAMP primer sets HiA1, HiC1, HiD1, HiE1, and HiF1 enabled real-time detection of various H. influenzae Types, and that these LAMP primer sets were excellent in terms of quantitative performance. Among others, when the HiA1 and HiF1 LAMP primer sets were used, an extremely short threshold time was obtained, and these primer sets were excellent in terms of detection promptness.

### INDUSTRIAL APPLICABILITY

The present invention provides: a method of detecting capsular serotype Haemophilus influenzae other than H. influenzae Type b (namely, H. influenzae Types a, c, d, e and f); and a primer set and a detection kit that can be used for the aforementioned detection method. The detection method of the present invention is extremely useful for clinical diagnoses and the subsequent treatments in that it is able to rapidly, simply and accurately perform typing of such capsular serotype Haemophilus influenzae other than the H. influenzae Type b according to a LAMP method.

### Sequence Listing Free Text

### SEQ ID NO: 1 Synthetic DNA

### SEQ ID NO: 2 Synthetic DNA

### SEQ ID NO: 3 Synthetic DNA

### SEQ ID NO: 4 Synthetic DNA

### SEQ ID NO: 5 Synthetic DNA

### SEQ ID NO: 6 Synthetic DNA

### SEQ ID NO: 7 Synthetic DNA

### SEQ ID NO: 8 Synthetic DNA

### SEQ ID NO: 9 Synthetic DNA

### SEQ ID NO: 10 Synthetic DNA

### SEQ ID NO: 11 Synthetic DNA

### SEQ ID NO: 12 Synthetic DNA

### SEQ ID NO: 13 Synthetic DNA

### SEQ ID NO: 14 Synthetic DNA

### SEQ ID NO: 15 Synthetic DNA

### SEQ ID NO: 16 Synthetic DNA

### SEQ ID NO: 17 Synthetic DNA

### SEQ ID NO: 18 Synthetic DNA

### SEQ ID NO: 19 Synthetic DNA

### SEQ ID NO: 20 Synthetic DNA

### SEQ ID NO: 21 Synthetic DNA

### SEQ ID NO: 22 Synthetic DNA

### SEQ ID NO: 23 Synthetic DNA

### SEQ ID NO: 24 Synthetic DNA

### SEQ ID NO: 25 Synthetic DNA

SEQ ID NO: 26 Synthetic DNA

1. A method of detecting Haemophilus influenzae Type a, which comprises: amplifying capsulation locus region II derived from Haemophilus influenzae Type a, using a LAMP primer set comprising one or more types of primers each having a nucleotide sequence that is identical to or complementary to a partial sequence in the nucleotide sequence region of the capsulation locus region II; and detecting the obtained amplification product.

2. The method according to claim 1, wherein the LAMP primer set consists of an FIP primer, a BIP primer, an F3 primer and a B3 primer designed from the nucleotide sequence region as shown in SEQ ID NO: 27 in the capsulation locus region II.

3. The method according to claim 2, wherein the LAMP primer set further comprises an LF primer and/or an LB primer as a loop primer(s).

4. The method according to claim 2, wherein the FIP primer is designed from a region ranging from bp 3216 to 3288 in the nucleotide sequence as shown in SEQ ID NO: 27.

5. The method according to claim 2, wherein the BIP primer is designed from a region ranging from bp 3305 to 3387 in the nucleotide sequence as shown in SEQ ID NO: 27.

6. The method according to claim 2, wherein the F3 primer is designed from a region ranging from bp 3197 to 3214 in the nucleotide sequence as shown in SEQ ID NO: 27.

7. The method according to claim 2, wherein the B3 primer is designed from a region ranging from bp 3408 to 3429 in the nucleotide sequence as shown in SEQ ID NO: 27.

8. The method according to claim 3, wherein the LF primer is designed from a region ranging from bp 3239 to 3263 in the nucleotide sequence as shown in SEQ ID NO: 27.

9. The method according to claim 3, wherein the LB primer is designed from a region ranging from bp 3340 to 3364, or from bp 3339 to 3362 in the nucleotide sequence as shown in SEQ ID NO: 27.

10. The method according to any one of claim 1, wherein the LAMP primer set is a combination of the nucleotide sequences described in the following (a), (b) or (c):
(a) a combination of the nucleotide sequences as shown in SEQ ID NOS: 1, 2, 3 and 4;
(b) a combination of the nucleotide sequences as shown in SEQ ID NOS: 1, 2, 3, 4, 5 and 6; or
(c) a combination of the nucleotide sequences as shown in SEQ ID NOS: 1, 2, 3, 4, 5 and 7.

11. A LAMP primer set for detecting Haemophilus influenzae Type a, which comprises a combination of the nucleotide sequences described in the following (a), (b) or (c):
(a) a combination of the nucleotide sequences as shown in SEQ ID NOS: 1, 2, 3 and 4;
(b) a combination of the nucleotide sequences as shown in SEQ ID NOS: 1, 2, 3, 4, 5 and 6; or
(c) a combination of the nucleotide sequences as shown in SEQ ID NOS: 1, 2, 3, 4, 5 and 7.

12. A kit for detecting Haemophilus influenzae Type a, which comprises the LAMP primer set according to claim 11.

13. A method of detecting Haemophilus influenzae Type c, which comprises: amplifying capsulation locus region II derived from Haemophilus influenzae Type c, using a LAMP primer set comprising one or more types of primers each having a nucleotide sequence that is identical to or complementary to a partial sequence in the nucleotide sequence region of the capsulation locus region II; and detecting the obtained amplification product.

14. The method according to claim 13, wherein the LAMP primer set consists of an FIP primer, a BIP primer, an F3 primer and a B3 primer designed from the nucleotide sequence region as shown in SEQ ID NO: 29 in the capsulation locus region II.

15. The method according to claim 14, wherein the FIP primer is designed from a region ranging from bp 64 to 140 in the nucleotide sequence as shown in SEQ ID NO: 29.

16. The method according to claim 14, wherein the BIP primer is designed from a region ranging from bp 141 to 219 in the nucleotide sequence as shown in SEQ ID NO: 29.

17. The method according to claim 14, wherein the F3 primer is designed from a region ranging from bp 42 to 61 in the nucleotide sequence as shown in SEQ ID NO: 29.

18. The method according to claim 14, wherein the B3 primer is designed from a region ranging from bp 229 to 252 in the nucleotide sequence as shown in SEQ ID NO: 29.

19. The method according to claim 13, wherein the LAMP primer set is a combination of the nucleotide sequences as shown in SEQ ID NOS: 8, 9, 10 and 11.

20. A LAMP primer set for detecting Haemophilus influenzae Type c, which comprises a combination of the nucleotide sequences as shown in SEQ ID NOS: 8, 9, 10 and 11.

21. A kit for detecting Haemophilus influenzae Type c, which comprises the LAMP primer set according to claim 20.

22. A method of detecting Haemophilus influenzae Type d, which comprises: amplifying capsulation locus region II derived from Haemophilus influenzae Type d, using a LAMP primer set comprising one or more types of primers each having a nucleotide sequence that is identical to or complementary to a partial sequence in the nucleotide sequence region of the capsulation locus region II; and detecting the obtained amplification product.

23. The method according to claim 22, wherein the LAMP primer set consists of an FIP primer, a BIP primer, an F3 primer and a B3 primer designed from the nucleotide sequence region as shown in SEQ ID NO: 30 in the capsulation locus region II.

24. The method according to claim 23, wherein the FIP primer is designed from a region ranging from bp 346 to 410 in the nucleotide sequence as shown in SEQ ID NO: 30.

25. The method according to claim 23, wherein the BIP primer is designed from a region ranging from bp 445 to 519 in the nucleotide sequence as shown in SEQ ID NO: 30.

26. The method according to claim 23, wherein the F3 primer is designed from a region ranging from bp 320 to 342 in the nucleotide sequence as shown in SEQ ID NO: 30.

27. The method according to claim 23, wherein the B3 primer is designed from a region ranging from bp 527 to 550 in the nucleotide sequence as shown in SEQ ID NO: 30.

28. The method according to claim 22, wherein the LAMP primer set is a combination of the nucleotide sequences as shown in SEQ ID NOS: 12, 13, 14 and 15.

29. A LAMP primer set for detecting Haemophilus influenzae Type d, which comprises a combination of the nucleotide sequences as shown in SEQ ID NOS: 12, 13, 14 and 15.

30. A kit for detecting Haemophilus influenzae Type d, which comprises the LAMP primer set according to claim 29.

31. A method of detecting Haemophilus influenzae Type e, which comprises: amplifying capsulation locus region II derived from Haemophilus influenzae Type e, using a LAMP primer set comprising one or more types of primers each having a nucleotide sequence that is identical to or complementary to a partial sequence in the nucleotide sequence region of the capsulation locus region II; and detecting the obtained amplification product.

32. The method according to claim 31, wherein the LAMP primer set consists of an FIP primer, a BIP primer, an F3 primer and a B3 primer designed from the nucleotide sequence region as shown in SEQ ID NO: 31 in the capsulation locus region II.

33. The method according to claim 32, wherein the FIP primer is designed from a region ranging from bp 608 to 667 in the nucleotide sequence as shown in SEQ ID NO: 31.

34. The method according to claim 32, wherein the BIP primer is designed from a region ranging from bp 687 to 770 in the nucleotide sequence as shown in SEQ ID NO: 31.

35. The method according to claim 32, wherein the F3 primer is designed from a region ranging from bp 582 to 599 in the nucleotide sequence as shown in SEQ ID NO: 31.

36. The method according to claim 32, wherein the B3 primer is designed from a region ranging from bp 781 to 798 in the nucleotide sequence as shown in SEQ ID NO: 31.

37. The method according to claim 31, wherein the LAMP primer set is a combination of the nucleotide sequences as shown in SEQ ID NOS: 16, 17, 18 and 19.

38. A LAMP primer set for detecting Haemophilus influenzae Type e, which comprises a combination of the nucleotide sequences as shown in SEQ ID NOS: 16, 17, 18 and 19.

39. A kit for detecting Haemophilus influenzae Type e, which comprises the LAMP primer set according to claim 38.

40. A method of detecting Haemophilus influenzae Type f, which comprises: amplifying capsulation locus region II derived from Haemophilus influenzae Type f, using a LAMP primer set comprising one or more types of primers each having a nucleotide sequence that is identical to or complementary to a partial sequence in the nucleotide sequence region of the capsulation locus region II; and detecting the obtained amplification product.

41. The method according to claim 40, wherein the LAMP primer set consists of an FIP primer, a BIP primer, an F3 primer and a B3 primer designed from the nucleotide sequence region as shown in SEQ ID NO: 33 in the capsulation locus region II.

42. The method according to claim 41, wherein the LAMP primer set further comprises an LF primer and/or an LB primer as loop primer(s).

43. The method according to claim 41, wherein the FIP primer is designed from a region ranging from bp 12086 to 12169 in the nucleotide sequence as shown in SEQ ID NO: 33.

44. The method according to claim 41, wherein the BIP primer is designed from a region ranging from bp 12184 to 12266 in the nucleotide sequence as shown in SEQ ID NO: 33.

45. The method according to claim 41, wherein the F3 primer is designed from a region ranging from bp 12063 to 12084 in the nucleotide sequence as shown in SEQ ID NO: 33.

46. The method according to claim 41, wherein the B3 primer is designed from a region ranging from bp 12281 to 12304 in the nucleotide sequence as shown in SEQ ID NO: 33.

47. The method according to claim 42, wherein the LF primer is designed from a region ranging from bp 12116 to 12139 in the nucleotide sequence as shown in SEQ ID NO: 33.

48. The method according to claim 42, wherein the LB primer is designed from a region ranging from bp 12210 to 12234, or from bp 12117 to 12139 in the nucleotide sequence as shown in SEQ ID NO: 33.

49. The method according to claim 40, wherein the LAMP primer set is a combination of the nucleotide sequences described in the following (a), (b) or (c):
(a) a combination of the nucleotide sequences as shown in SEQ ID NOS: 20, 21, 22 and 23;
(b) a combination of the nucleotide sequences as shown in SEQ ID NOS: 20, 21, 22, 23, 24 and 25; or
(c) a combination of the nucleotide sequences as shown in SEQ ID NOS: 20, 21, 22, 23, 24 and 26.

50. A LAMP primer set for detecting Haemophilus influenzae Type f, which comprises a combination of the nucleotide sequences described in the following (a), (b) or (c):
(a) a combination of the nucleotide sequences as shown in SEQ ID NOS: 20, 21, 22 and 23;
(b) a combination of the nucleotide sequences as shown in SEQ ID NOS: 20, 21, 22, 23, 24 and 25; or
(c) a combination of the nucleotide sequences as shown in SEQ ID NOS: 20, 21, 22, 23, 24 and 26.

51. A kit for detecting Haemophilus influenzae Type f, which comprises the LAMP primer set according to claim 50.
